(19) United States
(12) Patent Application Publication
(10) Pub. No.: US 2011/0184311 A1
(43) Pub. Date: Jul. 28, 2011

(54) Tissue Retrieval Device with Resilient Member

(76) Inventors: Shailendra K. Parihar, Mason, OH (US); Patrick J. Minnelli, Harrison, OH (US); Gregory W. Johnson, Milford, OH (US); Mark J. Bookbinder, Blue Ash, OH (US); Nabeel M. Jadeed, Cincinnati, OH (US); Frederick E. Shelton, IV, Hillsboro, OH (US)

(21) Appl. No.: 12/692,727

(22) Filed: Jan. 25, 2010

(51) Int. Cl. A61B 10/02; A61B 17/50

(52) U.S. Cl. 600/562; 606/114

(57) ABSTRACT

A specimen retrieval instrument comprises a tubular member, a resilient member, and a retrieval bag. The resilient member is associated with a distal end of the tubular member. The bag is secured to the resilient member. The resilient member is compressible such that the resilient member and bag fit within a hollow interior portion of the tubular member. A rod may advance the resilient member and bag out of the tubular member. Alternatively, the tubular member may comprise a sheath that is retractable to reveal the resilient member and bag. The resilient member may have a hoop, spiral, or rolled strip configuration. The resilient member may alternatively comprise telescoping arms. The resilient member opens the bag when the resilient member and bag are deployed relative to the tubular member. The resilient member may also unfurl the bag when the resilient member and bag are deployed.

### BACKGROUND

Endoscopic surgery (e.g., laparoscopy) is a procedure wherein surgery is performed through a series of small openings or incisions in a patient. This type of surgery may reduce or eliminate the need for large incisions and may change some otherwise open surgical procedures such as gall bladder removal to simple outpatient surgery. Consequently, the patient's recovery time may change from weeks to days. These types of surgeries may be used for repairing defects or for the removal of diseased tissue or organs from areas of the body such as the abdominal recess. In some of these procedures, biological material or tissue may be removed or excised from the body through a small opening such as an incision, a small natural orifice, or through a small diameter laparoscopic access port such as a trocar.

Various types of tissue retrieval pouches or bags have been developed to allow for the removal of tissue through a small opening, orifice, or port in an endoscopic surgical procedure. Various instruments have also been devised for introducing, opening, positioning, and closing tissue retrieval bags within a patient; and for removing the bags and enclosed tissue from the surgical site. Some exemplary retrieval bags and associated instruments are disclosed in U.S. Pat. No. 5,465,731, entitled “Specimen Retrieval Pouch and Method for Use,” issued Nov. 14, 1995, the disclosure of which is incorporated by reference herein; U.S. Pat. No. 5,480,404, entitled “Surgical Tissue Retrieval Instrument,” issued Jan. 2, 1996, the disclosure of which is incorporated by reference herein; U.S. Pat. No. 5,647,372, entitled “Specimen Retrieval Pouch and Method for Use,” issued Jul. 15, 1997, the disclosure of which is incorporated by reference herein; U.S. Pat. No. 5,971,995, entitled “Surgical Pouch Instrument,” issued Oct. 26, 1999, the disclosure of which is incorporated by reference herein; and U.S. Pat. No. 6,409,733, entitled “Specimen Retrieval Bag,” issued Jun. 25, 2002, the disclosure of which is incorporated by reference herein.

While a variety of tissue retrieval devices have been made and used, it is believed that no one prior to the inventor(s) has made or used an invention as described herein.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings. In the drawings, like numerals represent like elements throughout the several views.

FIG. 1 is a perspective view of an exemplary specimen retrieval instrument, with a sheath in a distal position to contain a retrieval bag, and with a handle assembly omitted.

FIG. 2 is a perspective view of the specimen retrieval instrument of FIG. 1, with the sheath in a proximal position to reveal the retrieval bag.

FIG. 3 is a top view of the distal end of the specimen retrieval instrument of FIG. 1, with the sheath in the distal position and in cross section, and with the retrieval bag omitted.

FIG. 4 is a top view of the distal end of the specimen retrieval instrument of FIG. 1, with the sheath in the proximal position and with the retrieval bag opened.

FIG. 5 is a front view of another exemplary specimen retrieval instrument having a resilient loop and shown in an undeployed position.

FIG. 6 is a front view of the specimen retrieval instrument of FIG. 5, shown in an intermediate, position between undeployed and fully deployed.

FIG. 7 is a front view of the specimen retrieval instrument of FIG. 5, shown in a fully deployed position.

FIG. 8 is a top view of the distal end of the specimen retrieval instrument of FIG. 5, shown in the undeployed position.

FIG. 9 is a top view of the distal end of the specimen retrieval instrument of FIG. 5, shown in the fully deployed position.

FIG. 10 is a top view of the distal end of another exemplary specimen retrieval instrument, with a sheath cross section and in a distal position to contain a retrieval bag and resilient loop, with the retrieval bag omitted.

FIG. 11 is a top view of the distal end of the specimen retrieval instrument of FIG. 10, with the sheath in cross-section and in a proximal position, with a resilient loop in a partially deployed position, and with the retrieval bag omitted.

FIG. 12 is a top view of the distal end of the specimen retrieval instrument of FIG. 10, with the sheath in cross-section and in the proximal position, with a resilient loop in a fully deployed position, and with the retrieval bag omitted.

FIG. 13 is a side view of the distal end of the specimen retrieval instrument of FIG. 10, with the sheath in the proximal position and with the resilient loop and retrieval bag in a fully deployed position.

FIG. 14 is a perspective view of another exemplary specimen retrieval instrument, with a sheath in a distal position to contain a retrieval bag and telescoping arm.

FIG. 15 is a perspective view of the specimen retrieval instrument of FIG. 14, with the sheath in a proximal position to reveal the retrieval bag and telescoping aim and with the retrieval bag and telescoping arm in a collapsed configuration.

FIG. 16 is a perspective view of the specimen retrieval instrument of FIG. 14, with the sheath in the proximal position and with the retrieval bag and telescoping arm in an expanded configuration.

FIG. 17 is a top view of the distal end of the specimen retrieval instrument of FIG. 14, with the retrieval bag and telescoping arm in the collapsed configuration.

FIG. 18 is a side view of the distal portion of another exemplary specimen retrieval instrument having a spiral arm shown in an undeployed position within an introducer tube, which is shown in cross section.

FIG. 19 is a side view of the distal portion of the specimen retrieval instrument of FIG. 18, shown in the deployed position, with the introducer tube in cross section.

FIG. 20 is a perspective view of the distal portion of another exemplary specimen retrieval instrument having a retrieval bag with an unrolling stay, shown in a deployed position with the retrieval bag open.

FIG. 21 is a front view of the stay of FIG. 20, shown in a rolled position prior to deployment of the retrieval bag.

FIG. 22 is a perspective view of the distal portion of another exemplary specimen retrieval instrument having a twisted expanding retrieval bag and shown in a deployed position with the retrieval bag closed.

FIG. 23 is a perspective view of the distal portion of the specimen retrieval instrument of FIG. 22, shown in a deployed position with the retrieval bag partially open.

FIG. 24 is a perspective view of the distal portion of the specimen retrieval instrument of FIG. 21, shown in a deployed position with the retrieval bag fully open.

FIG. 25 is a side view of another exemplary specimen retrieval instrument.

The drawings are not intended to be limiting in any way, and it is contemplated that various embodiments of the invention may be carried out in a variety of other ways, including those not necessarily depicted in the drawings. The accompanying drawings incorporated in, and forming a part of, the specification illustrate several aspects of the present invention, and together with the description serve to explain the principles of the invention; it being understood, however, that this invention is not limited to the precise arrangements shown.

### DETAILED DESCRIPTION

The following description of certain examples should not be used to limit the scope of the present invention. Other features, aspects, and advantages of the versions disclosed herein will become apparent to those skilled in the art from the following description, which is by way of illustration, one of the best modes contemplated for carrying out the invention. As will be realized, the versions described herein are capable of other different and obvious aspects, all without departing from the invention. Accordingly, the drawings and descriptions should be regarded as illustrative in nature and not restrictive.

I. Exemplary Retrieval Bag Deployment

When designing a specimen retrieval instrument, an area to consider is the modes used to deploy a retrieval bag from the instrument once the instrument has been positioned within a patient. The following section will discuss several deployment modes that may include use of a resilient loop, a pivoting loop, or telescoping arm. Based on the teachings herein, other deployment modes will be apparent to those of ordinary skill in the art.

A. Exemplary Resilient Loop

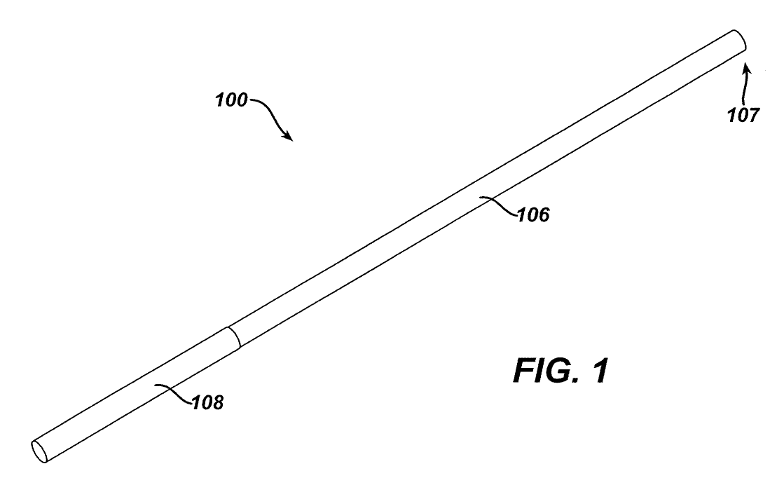

Referring to FIGS. 1-4, an exemplary specimen retrieval instrument 100 is shown.

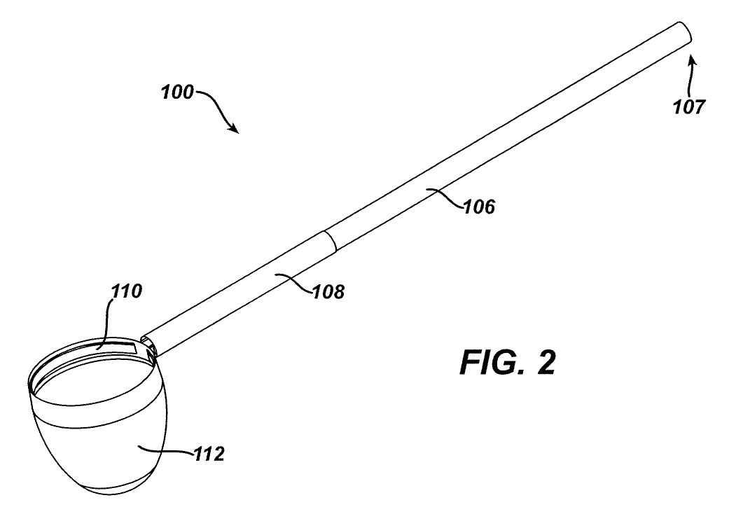

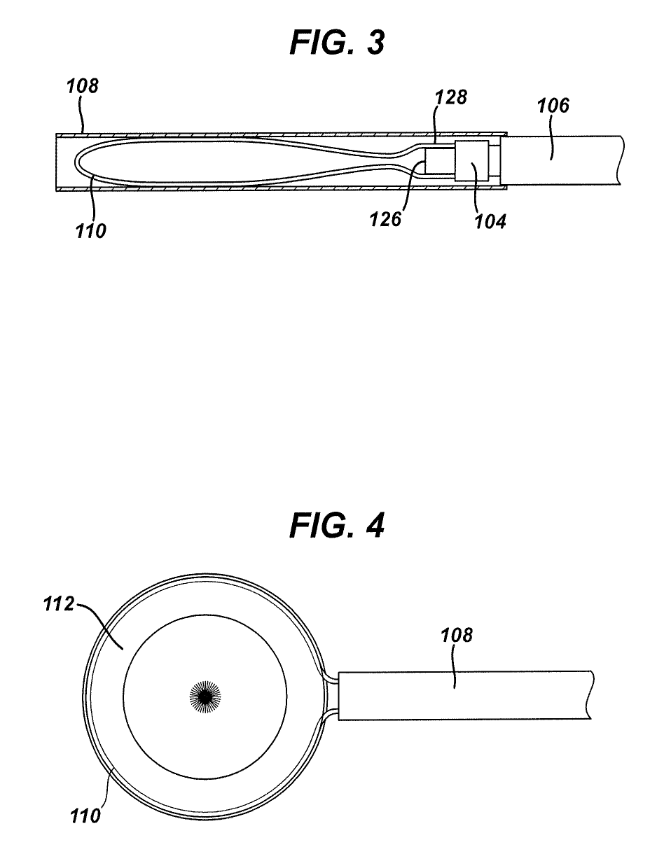

Specimen retrieval instrument 100 includes a rod 104, an introducer tube 106, a sheath 108, a loop 110, and a retrieval bag 112. Rod 104 is fixedly positioned within introducer tube 106 in the present example. However, in some other versions as will be described in greater detail below, rod 104 may me configured to reciprocate within introducer tube 106. Sheath 108 is configured to translate relative to introducer tube 106. In particular, and as will also be described in greater detail below, sheath 108 is translatable from a distal position (FIGS. 1 and 3) to a proximal position (FIGS. 2 and 4). Together, introducer tube 106 and sheath 108 are configured to fit within the insertion passageway defined by a device such as a trocar. By way of example only, introducer tube 106 and sheath 108 may present an outer diameter that is between approximately 5 mm (inclusive) and approximately 15 mm (inclusive) (e.g., approximately 10 mm, etc.). Alternatively, these components may have any other suitable dimensions. Loop 110 carries retrieval bag 112, and is resiliently biased to expand from a compressed configuration (FIGS. 1 and 3) to an expanded configuration (FIGS. 2 and 4), as will also be described in greater detail below.

FIGS. 1 and 3 show specimen retrieval instrument 100 in an undeployed configuration. In this configuration, sheath 108 is in a distal position, substantially enclosing resilient loop 110 and retrieval bag 112. When specimen retrieval instrument 100 is in the undeployed configuration, specimen retrieval instrument 100 is ready to be inserted within a patient. By way of example only, specimen retrieval instrument 100 may be inserted into a patient via a trocar or other type of access port device, via an incision, via a natural orifice, and/or in any other suitable fashion. Introducer tube 106 has a substantially cylindraceous configuration in the present example. However, it should be understood that introducer tube 106 may have any suitable configuration. In addition, it should be understood that any suitable type of handle assembly may be provided at the proximal end of introducer tube 106 and/or sheath 108. By way of example only, such a handle assembly may have one or more finger grips, thumb rings, or any other suitable structures, features, or configurations. Such a handle assembly may be configured to facilitate proximal translation of sheath 108 relative to introducer tube 106. In addition or in the alternative, one or more cables, strings, rods, or other features may be operable to provide proximal translation of sheath 108 relative to introducer tube 106.

FIGS. 2 and 4 show specimen retrieval instrument 100 in a deployed configuration. In this configuration, sheath 108 is in a proximal position, substantially revealing resilient loop 110 and retrieval bag 112. In the present example, specimen retrieval instrument 100 is transitioned from the undeployed configuration to the deployed configuration after the distal end of specimen retrieval instrument 100 has been inserted within a patient. To transition specimen retrieval instrument from the undeployed configuration to the deployed configuration, sheath 108 is retracted proximally relative to introducer tube 106. It should be understood that such proximal retraction of sheath 108 may be accomplished in a variety of ways. By way of example only, sheath 108 may have a length selected such that a proximal portion of sheath 108 protrudes proximally relative to a trocar or other access port when the distal end of specimen retrieval instrument 100 is inserted in a patient. Thus, such a proximal portion of sheath 108 may be manipulated by a surgeon or other user externally relative to the patient during a surgical procedure. A handle, grip, or other structural feature may be provided at the proximal end of sheath 108 to facilitate such extracorporeal manipulability of sheath 108. In addition or in the alternative, a string, cable, or other feature may be coupled with sheath 108 and may be operable to retract sheath 108 proximally. In addition or in the alternative, a feature that is operable to retract sheath 108 proximally may extend within the interior of introducer tube 106, in addition to or as an alternative to extending along the exterior of introducer tube 106.

In some alternative versions, sheath 108 is removable from introducer tube 106 by pulling sheath 108 off of introducer tube 106 in a distal direction; rather than providing proximal retractability of sheath 108. By way of example only, sheath 108 may be configured like a sleeve or sock, and a separate instrument (e.g., conventional tissue graspers, etc.) may be used to pull sheath 108 distally off of resilient loop 110 and retrieval bag 112. In addition or in the alternative, sheath 108 may have a perforation or other weakening feature that may be breached to remove sheath 108 by tearing sheath 108 away from introducer tube 106. As yet another merely illustrative alternative, sheath 108 may be formed of an environmentally sensitive material. For instance, sheath 108 may be configured to dissolve or substantially weaken in the presence of bodily fluid, certain temperatures, or other environmental parameters that may be associated with the interior of a patient. Still other various ways in which sheath 108 may be configured and/or operable will be apparent to those of ordinary skill in the art in view of the teachings herein. Furthermore, sheath 108 may even be omitted in some versions.

As noted above, loop 110 is resiliently biased to maintain a substantially circular shape when not constricted by other components. Retrieval bag 112 is associated with loop 110 such that retrieval bag 112 opens when loop 110 assumes its substantially circular shape. For example, as shown in FIG. 4, retrieval bag 112 may be connected to an outer perimeter of loop 110. By way of example only, loop 110 may be inserted through one or more sleeves, slots, pockets, loops, slits, etc., formed in retrieval bag 112. Various suitable ways in which retrieval bag 112 may be secured to loop 110 will be apparent to those of ordinary skill in the art in view of the teachings herein. Furthermore, retrieval bag 112 may be wrapped about loop 110 when loop 110 and retrieval bag 112 are located within sheath 108. In some versions, after deployment, rotation of a knob, thumb ring, or other feature may rotate rod 104, which may further cause retrieval bag 112 to unwrap from loop 110. In some other versions, retrieval bag 112 may unwrap by itself (e.g., in response to its own resilient bias, in response to gravity, etc.) during opening of loop 110 upon deployment. In addition or in the alternative, an additional device (e.g., conventional tissue graspers, etc.) may be used to assist in unfurling, unwrapping, opening, etc. of retrieval bag 112. It should also be understood that loop 110 may be resiliently biased to assume a variety of other shapes, and that such shapes need not necessarily be circular in all versions.

Referring to FIGS. 3 and 4, a more detailed view of the distal portion of specimen retrieval instrument 100 is shown. As shown, loop 110 is connected to distal end 126 of rod 104. In the present example, the connection of loop 110 to rod 104 is achieved by loop 110 having parallel proximal ends 128 that secure to respective sides of distal end 126 of rod 104 by the use of securing pins (not shown) or other suitable attachment means. As noted above, rod 104 is fixedly positioned within introducer tube 106 in the present example. Accordingly, loop 110 and retrieval bag 112 are fixedly positioned relative to introducer tube 106 in the present example. However, it should be understood that these components may be substituted with various other components or structures; and that these components may have a variety of alternative relationships with each other. By way of example only, in some versions rod 104 is configured to translate relative to introducer tube 106. In some such versions, rod 104 is movable to a proximal position where loop 110 and retrieval bag 112 are located within the hollow interior defined by introducer tube 106. In some such versions, rod 104 is distally translatable relative to introducer tube 106, to reveal loop 110 and retrieval bag 112 from the open distal end of introducer tube 106. In other words, in some versions where rod 104 is translatable relative to introducer tube 106, introducer tube 106 may play a role similar to that described above for sheath 108. Various suitable configurations for handle assemblies that may facilitate translation of rod 104 relative to introducer tube 106 will be apparent to those of ordinary skill in the art in view of the teachings herein.

In use, specimen retrieval instrument 100 may initially have the arrangement shown in FIGS. 1 and 3, where sheath 108 is at a distal position. In this arrangement, the distal portion of specimen retrieval instrument 100 may be inserted within a patient through a suitable incision opening or access port created by a trocar or some other device. Once positioned within the patient, sheath 108 may be retracted proximally. This action reveals resilient loop 110 and retrieval bag 112. With resilient loop 110 being so freed from the confines of sheath 108, the resilient bias of loop 110 causes loop to expand to the configuration shown in FIGS. 2 and 4, thereby opening retrieval bag. Once retrieval bag 112 has been opened, one or more tissue specimens, etc., may be placed within retrieval bag 112. Once a specimen has been placed within retrieval bag 112, sheath 108 may be advanced distally relative to introducer tube 106, with introducer tube 106 maintaining a substantially constant position relative to the patient (or introducer tube 106 may be retracted proximally relative to sheath 108, with sheath 108 maintaining a substantially constant position relative to the patient). Such a motion may thus cause sheath 108 to at least substantially envelop loop 110 once again. In particular, such engagement between sheath 108 and loop 110 may cause loop 110 to transition back toward a substantially collapsed configuration. Overcoming the resilient bias of loop 110 in this way may permits loop 110 and retrieval bag 112 to close, and in some versions for a proximal portion of loop 110 to be constrained within a distal portion of sheath 108. With retrieval bag 112 closed, specimen retrieval instrument 100 is now ready to be removed from the patient. Of course, there does not necessarily have to be any relative movement between sheath 108 and introducer tube 106 after a specimen has been placed in retrieval bag 112 and before specimen retrieval instrument 100 is removed from the patient.

In some versions, specimen retrieval instrument 100 is configured such that retrieval bag 112 may be removed from specimen retrieval instrument 100 while retrieval bag 112 is within the patient. Some such versions facilitate removal of retrieval bag 112 separate from removal of the other components of specimen retrieval instrument 100. In some versions, this may be accomplished by, among other ways, retrieval bag 112 being removable from loop 110. For instance, in some versions specimen retrieval instrument 100 may include a closure string connected to retrieval bag 112 and having a slipknot attachment to rod 104, introducer tube 106, or sheath 108. Pulling the slipknot loose and retracting introducer tube 106 may permit detachment of retrieval bag 112 and the closure string from the other components of specimen retrieval instrument 100. In some such versions, a user may pull the closure string to close retrieval bag 112. By way of example only, such a closure mechanism may be configured in accordance with the teachings of U.S. Pat. No. 6,409,733, entitled “Specimen Retrieval Bag,” issued Jun. 25, 2002, which is incorporated herein by reference. Still in other versions, it may be feasible to incorporate a closure string with retrieval bag 112, and to release retrieval bag 112 from specimen retrieval instrument 100 such that retrieval bag 112 may be removed from the patient separate from other components of specimen retrieval instrument 100.

While the above description provides adequate disclosure to enable one of ordinary skill in the art to make and use specimen retrieval instrument 100, based on the teachings herein, those of ordinary skill in the art will appreciate that various other modifications may provide additional features or functionality. For example, while the above versions were described as having both a sheath 108 and an introducer tube 106, in some versions introducer tube 106 may be omitted; while in other versions sheath 108 may be omitted. In some versions modifications may include rod 104 or introducer tube 106 having features operable with features of sheath 108 or other components to prevent inadvertent retraction of sheath 108 and premature opening of loop 110 and retrieval bag 112. For example, rod 104 may include a lock or stop that may be released once a user is ready to open loop 110 and retrieval bag 112. Still in other versions, modifications may include rod 104 having features operable with features of sheath 108 or other components to prevent inadvertent advancement of sheath 108 after initial deployment of loop 110 and opening of retrieval bag 112. For example, rod 104 may be associated with a resilient tab configured to engage a corresponding opening in a sidewall of sheath 108 upon deployment of loop 110. The engagement between the resilient tab and the opening in the sidewall of sheath 108 may act as a locking mechanism that prevents inadvertent advancement of sheath 108, and therefore inadvertent premature closure of loop 110 and retrieval bag 112. Other ways in which inadvertent retraction and/or advancement of sheath 108 may be avoided through various features of specimen retrieval instrument 100 will be apparent to those of ordinary skill in the art in view of the teachings herein.

In some versions, modifications may include providing loop 110 in various sizes with various sized retrieval bags 112. In other words, loop 110 and/or retrieval bag 112 may be modular in nature, and may come in a kit having loops 110 and/or retrieval bags 112 in various sizes. In some such versions, the desired sized loop 110 and retrieval bag 112 may be attached to rod 104 prior to insertion of specimen retrieval instrument 100 within a patient.

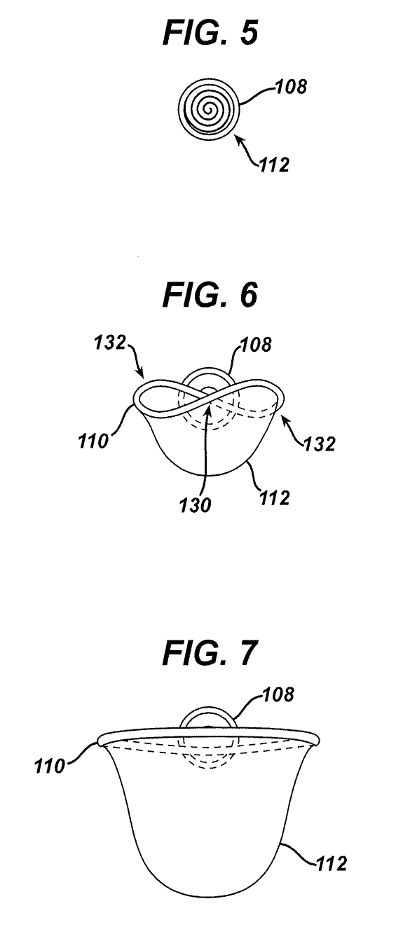

Referring to FIGS. 5-9, in some versions, specimen retrieval instrument 100 may be modified such that loop 110 is twisted or otherwise folded within sheath 108 when in the undeployed position. When loop 110 is deployed, by distal extension and/or proximal retraction of other components as described above, loop 110 unfolds and returns to its normal circular shape.

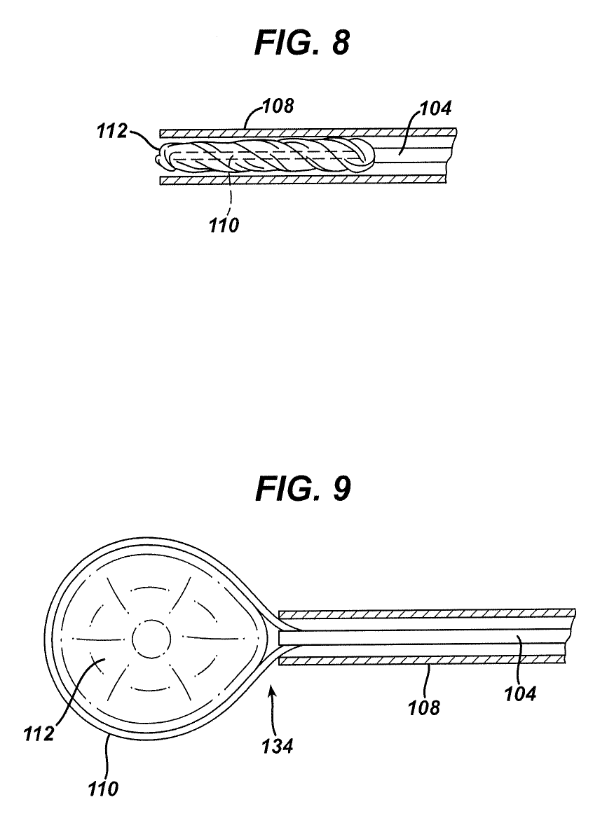

As shown in FIGS. 5 and 8, loop 110 and retrieval bag 112 are located within sheath 108 of specimen retrieval instrument 100 when specimen retrieval instrument is in an undeployed position. As shown in FIGS. 7 and 9, after deployment, loop 110 and retrieval bag 112 extend from sheath 108 and are fully open. While folding of loop 110 may be accomplished in a variety of manners, one such version may include loop 110 being twisted about a longitudinal axis defined by rod 104, introducer tube 106, and sheath 108, as shown in FIGS. 5 and 8. Such a configuration may result in loop 110 resiliently untwisting itself as loop 110 is being revealed by proximal retraction of sheath 108. FIG. 6 shows loop 110 in a transition stage between being fully twisted within sheath 108 (FIGS. 5 and 8) and being untwisted and fully open (FIGS. 7 and 9). In particular, FIG. 6 shows loop 110 having a figure-eight configuration at this transition stage, with such a figure-eight being formed by outer loop portions 132 and an intersecting point 130. One portion of loop 110 at intersecting point 130 of the figure-eight may be connected with rod 104. When folded and located within sheath 108, intersecting point 130 of the figure-eight is positioned most proximal, while outer loop portions 132 of the figure-eight fold distally and are positioned most distal. Upon deployment of loop 110, loop 110 unfolds, returning to its normal circular loop orientation and retrieval bag 112 opens.

Once retrieval bag 112 is ready to be removed from the patient, the various modes for closing and removing retrieval bag 112 described above may be adapted for use with versions incorporating folded/twisted loop 110. For example, in some versions, retrieval bag 112 may be separable from loop 110 by use of a closure string as discussed above. In other versions, loop 110 may be naturally biased to fold to a closed position when sheath 108 contacts proximal portion 134 of deployed loop 110 with a sufficient force.

While the above description provides adequate disclosure to enable one of ordinary skill in the art to make and use specimen retrieval instrument 100 incorporating a folded loop, based on the teachings herein, those of ordinary skill in the art will appreciate that various other modifications may provide additional features or functionality. For example, numerous fold patterns may be suitable for use in versions incorporating a folded loop 110. For example, some fold patterns, materials, and/or section configurations, etc., may create more or less potential energy within loop 110 that will influence how rapidly loop 110 unfolds and opens upon deployment. Still various other suitable features, components, configurations, and operabilities that may be incorporated into specimen retrieval instrument 100 will be apparent to those of ordinary skill in the art in view of the teachings herein.

B. Exemplary Pivoting Loop

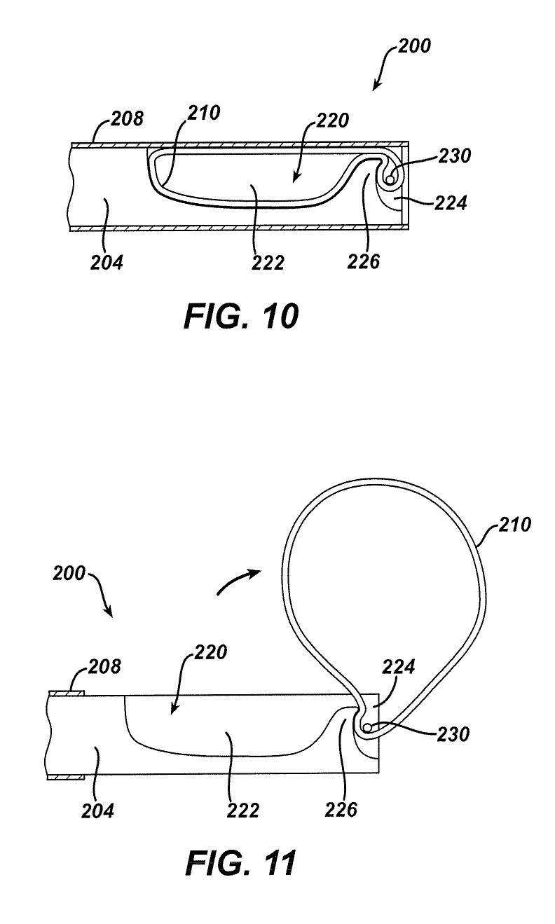

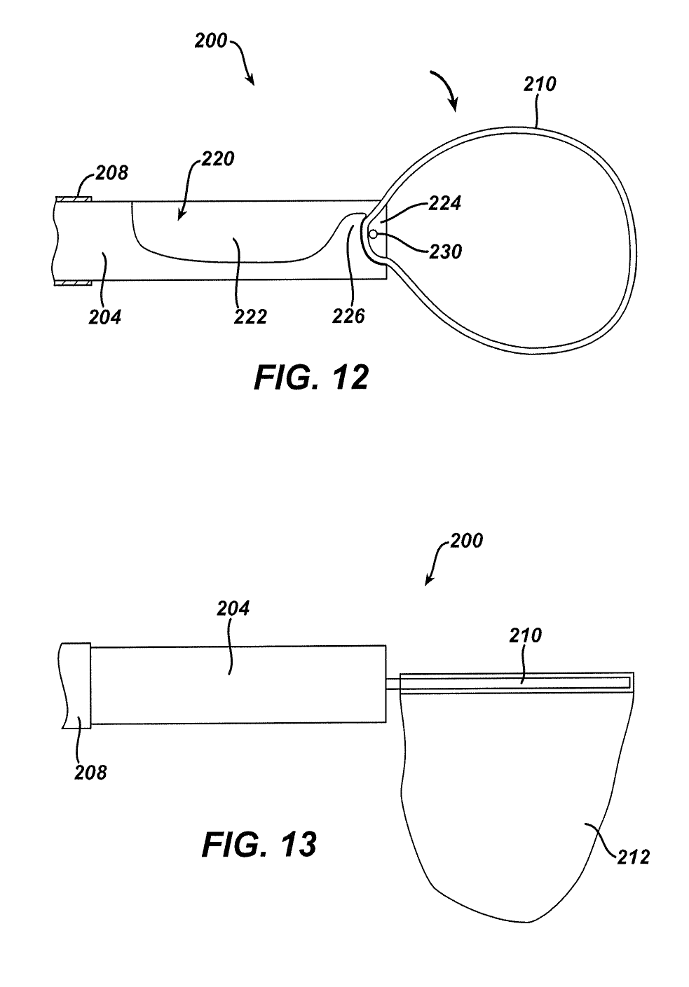

FIGS. 10-13 show the distal end of an exemplary alternative specimen retrieval instrument 200. Specimen retrieval instrument 200 includes a rod 204, sheath 208, loop 210, and retrieval bag 212. Some versions may also include an introducer tube (e.g., like introducer tube 106 described above, etc.) coaxially positioned between rod 204 and sheath 208. Sheath 208 is configured to translate relative to rod 204. In particular, and as will also be described in greater detail below, sheath 208 is translatable from a distal position (FIG. 10) to a proximal position (FIGS. 12-13). Together, rod 204 and sheath 208 are configured to fit within the insertion passageway defined by a device such as a trocar. By way of example only, rod 204 and sheath 208 may present an outer diameter that is between approximately 5 mm (inclusive) and approximately 15 mm (inclusive). Alternatively, these components may have any other suitable dimensions. Loop 210 carries retrieval bag 212, and is resiliently biased to expand from a compressed configuration (FIG. 10) to an expanded configuration (FIGS. 12-13), as will also be described in greater detail below.

FIG. 10 shows specimen retrieval instrument 200 in an undeployed configuration. In this configuration, sheath 208 is in a distal position, substantially enclosing resilient loop 210 and retrieval bag 212. When specimen retrieval instrument 200 is in the undeployed configuration, specimen retrieval instrument 200 is ready to be inserted within a patient. By way of example only, specimen retrieval instrument 200 may be inserted into a patient via a trocar or other type of access port device, via an incision, via a natural orifice, and/or in any other suitable fashion. Rod 204 has a substantially cylindraceous configuration in the present example. However, it should be understood that rod 204 may have any suitable configuration. In addition, it should be understood that any suitable type of handle assembly may be provided at the proximal end of rod 204 and/or sheath 208. By way of example only, such a handle assembly may have one or more finger grips, thumb rings, or any other suitable structures, features, or configurations.

FIGS. 12-13 show specimen retrieval instrument 200 in a deployed configuration, while FIG. 11 shows specimen retrieval instrument at a transitional stage between the undeployed configuration and the deployed configuration. In this deployed configuration, sheath 208 is in a proximal position, substantially revealing resilient loop 210 and retrieval bag 212. In the present example, specimen retrieval instrument 200 is transitioned from the undeployed configuration to the deployed configuration after the distal end of specimen retrieval instrument 200 has been inserted within a patient. To transition specimen retrieval instrument from the undeployed configuration to the deployed configuration, sheath 208 is retracted proximally relative to rod 204. It should be understood that such proximal retraction of sheath 208 may be accomplished in a variety of ways. By way of example only, sheath 208 may have a length selected such that a proximal portion of sheath 208 protrudes proximally relative to a trocar or other access port when the distal end of specimen retrieval instrument 200 is inserted in a patient. Thus, such a proximal portion of sheath 208 may be manipulated by a surgeon or other user externally relative to the patient during a surgical procedure. A handle, grip, or other structural feature may be provided at the proximal end of sheath 208 to facilitate such extracorporeal manipulability of sheath 208. In addition or in the alternative, a string, cable, or other feature may be coupled with sheath 208 and may be operable to retract sheath 208 proximally. In addition or in the alternative, a feature that is operable to retract sheath 208 proximally may extend within an interior portion of rod 204, in addition to or as an alternative to extending along the exterior of rod 204.

In some alternative versions, sheath 208 is removable from rod 204 by pulling sheath 208 off of rod 204 in a distal direction; rather than providing proximal retractability of sheath 208. By way of example only, sheath 208 may be configured like a sleeve or sock, and a separate instrument (e.g., conventional tissue graspers, etc.) may be used to pull sheath 208 distally off of resilient loop 210 and retrieval bag 212. In addition or in the alternative, sheath 208 may have a perforation or other weakening feature that may be breached to remove sheath 208 by tearing sheath 208 away from rod 204. As yet another merely illustrative alternative, sheath 208 may be formed of an environmentally sensitive material. For instance, sheath 208 may be configured to dissolve or substantially weaken in the presence of bodily fluid, certain temperatures, or other environmental parameters that may be associated with the interior of a patient. Still other various ways in which sheath 208 may be configured and/or operable will be apparent to those of ordinary skill in the art in view of the teachings herein. Furthermore, sheath 208 may even be omitted in some versions.

As noted above, loop 210 is resiliently biased to maintain a substantially circular shape when not constricted by other components. Retrieval bag 212 is associated with loop 210 such that retrieval bag 212 opens when loop 210 assumes its substantially circular shape. For example, as shown in FIG. 13, retrieval bag 212 may be connected to an outer perimeter of loop 210. By way of example only, loop 210 may be inserted through one or more sleeves, slots, pockets, loops, slits, etc., formed in retrieval bag 212. Various suitable ways in which retrieval bag 212 may be secured to loop 210 will be apparent to those of ordinary skill in the art in view of the teachings herein. Furthermore, retrieval bag 212 may be wrapped about loop 210 when loop 210 and retrieval bag 212 are located within sheath 208. In some versions, after deployment, rotation of a knob, thumb ring, or other feature may rotate rod 204, which may further cause retrieval bag 212 to unwrap from loop 210. In some other versions, retrieval bag 212 may unwrap by itself (e.g., in response to its own resilient bias, in response to gravity, etc.) during opening of loop 210 upon deployment. In addition or in the alternative, an additional device (e.g., conventional tissue graspers, etc.) may be used to assist in unfurling, unwrapping, opening, etc. of retrieval bag 212. It should also be understood that loop 210 may be resiliently biased to assume a variety of other shapes, and that such shapes need not necessarily be circular in all versions.

As shown in FIGS. 10-12, the distal end of rod 204 includes a recess 220. Recess 220 includes a proximal portion 222 and a distal portion 224. A camming projection 226 is longitudinally positioned between proximal portion 222 and distal portion 224. Recess 220 is formed transversely in the distal end of rod 204. In some versions, recess 220 is formed as a slot that does not angularly extend a significant degree about the longitudinal axis of rod 204 (viewed from the distal end of rod 204 toward the proximal end of rod 204). In some other versions, recess 220 extends approximately 90° about the longitudinal axis of rod 204 (viewed from the distal end of rod 204 toward the proximal end of rod 204). Alternatively, recess 220 may angularly extend approximately 180° about the longitudinal axis of rod 204 or to any other suitable degree. Proximal portion 222 of recess 220 is configured to receive compressed loop 210, as shown in FIG. 10. In addition, while not shown in FIG. 10, proximal portion 222 of recess 220 is configured to receive retrieval bag 212, which may be wrapped about compressed loop 210, folded, twisted, and/or otherwise packed within proximal portion 222 of recess 220 with loop 210.

A pin 230 extends upwardly from distal portion 224 of recess 220 in the present example. Loop 210 is substantially secured between pin 230 and camming projection 226 in the present example, such that pin 230 and camming projection 226 together substantially secure loop 210 to rod 204. A cap (not shown) or other component/feature may be provided at the top of pin 230 to further secure loop 210. As can be seen from FIGS. 10-12, loop 210 is configured to resiliently transition from a compressed configuration to an expanded configuration. As part of this transition, portions of loop 210 rotate about pin 230. Camming projection 226 of the present example is shaped similar to the dorsal fin of a dolphin, and is configured to assist loop 210 in substantially maintaining the orientation shown in FIG. 12 when loop 210 is in the expanded configuration while also permitting loop 210 to be bent rearwardly to achieve the compressed configuration shown in FIG. 10. Of course, camming projection 226 may have a variety of alternative configurations as desired. Furthermore, camming projection 226 may even be omitted in some versions.

In the present example, once the distal end of specimen retrieval instrument 200 is suitably inserted in a patient, the position of rod 204 relative to the patient remains substantially constant while sheath 208 is retracted relative to rod 204 and relative to the patient to reveal loop 210 and retrieval bag 212. In some other versions, once the distal end of specimen retrieval instrument 200 is suitably inserted in a patient, the position of sheath 208 relative to the patient remains substantially constant while rod 204 is advanced relative to sheath 208 and relative to the patient to reveal loop 210 and retrieval bag 212. Various suitable configurations for handle assemblies that may facilitate translation of rod 204 relative to sheath 208 will be apparent to those of ordinary skill in the art in view of the teachings herein.

In use, specimen retrieval instrument 200 may initially have the arrangement shown in FIG. 10, where sheath 208 is at a distal position. In this arrangement, the distal portion of specimen retrieval instrument 200 may be inserted within a patient through a suitable incision opening or access port created by a trocar or some other device. Once positioned within the patient, sheath 208 may be retracted proximally. This action reveals resilient loop 210 and retrieval bag 212. With resilient loop 210 being so freed from the confines of sheath 208, the resilient bias of loop 210 causes loop 210 to expand and rotate to the configuration shown in FIGS. 12-13, thereby opening retrieval bag 212. Once retrieval bag 212 has been opened, one or more tissue specimens, etc., may be placed within retrieval bag 212. Once a specimen has been placed within retrieval bag 212, sheath 208 may be advanced distally relative to rod 104, with rod 104 maintaining a substantially constant position relative to the patient (or rod 104 may be retracted proximally relative to sheath 208, with sheath 208 maintaining a substantially constant position relative to the patient). Such a motion may thus cause sheath 208 to at least substantially envelop loop 210 once again. In particular, such engagement between sheath 208 and loop 210 may cause loop 210 to transition back toward a substantially collapsed configuration. Overcoming the resilient bias of loop 210 in this way may permits loop 210 and retrieval bag 212 to close, and in some versions for a proximal portion of loop 210 to be constrained within a distal portion of sheath 208. With retrieval bag 212 closed, specimen retrieval instrument 200 is now ready to be removed from the patient. Of course, there does not necessarily have to be any relative movement between sheath 208 and rod 204 after a specimen has been placed in retrieval bag 212 and before specimen retrieval instrument 200 is removed from the patient.

In some versions, specimen retrieval instrument 200 is configured such that retrieval bag 212 may be removed from specimen retrieval instrument 200 while retrieval bag 212 is within the patient. Some such versions facilitate removal of retrieval bag 112 separate from removal of the other components of specimen retrieval instrument 200. In some versions, this may be accomplished by, among other ways, retrieval bag 212 being removable from loop 210. For instance, in some versions specimen retrieval instrument 200 may include a closure string connected to retrieval bag 212 and having a slipknot attachment to rod 204 or sheath 208. Pulling the slipknot loose and retracting rod 204 may permit detachment of retrieval bag 212 and the closure string from the other components of specimen retrieval instrument 200. In some such versions, a user may pull the closure string to close retrieval bag 212. By way of example only, such a closure mechanism may be configured in accordance with the teachings of U.S. Pat. No. 6,409,733, entitled “Specimen Retrieval Bag,” issued Jun. 25, 2002, which is incorporated herein by reference. Still in other versions, it may be feasible to incorporate a closure string with retrieval bag 212, and to release retrieval bag 212 from specimen retrieval instrument 200 such that retrieval bag 212 may be removed from the patient separate from other components of specimen retrieval instrument 200.

While the above description provides adequate disclosure to enable one of ordinary skill in the art to make and use specimen retrieval instrument 200, based on the teachings herein, those of ordinary skill in the art will appreciate that various other modifications may provide additional features or functionality. For example, in some versions modifications may include rod 204 having features operable with features of sheath 208 or other components to prevent inadvertent retraction of sheath 208 and premature opening of loop 210 and retrieval bag 212. For example, rod 204 may include a lock or stop that may be released once a user is ready to open loop 210 and retrieval bag 212. Still in other versions, modifications may include rod 204 having features operable with features of sheath 208 or other components to prevent inadvertent advancement of sheath 208 after initial deployment of loop 210 and opening of retrieval bag 212. For example, rod 204 may be associated with a resilient tab configured to engage a corresponding opening in a sidewall of sheath 208 upon deployment of loop 210. The engagement between the resilient tab and the opening in the sidewall of sheath 208 may act as a locking mechanism that prevents inadvertent advancement of sheath 208, and therefore inadvertent premature closure of loop 210 and retrieval bag 212. Other ways in which inadvertent retraction and/or advancement of sheath 208 may be avoided through various features of specimen retrieval instrument 200 will be apparent to those of ordinary skill in the art in view of the teachings herein.

In some versions, modifications may include providing loop 210 in various sizes with various sized retrieval bags 212. In other words, loop 210 and/or retrieval bag 212 may be modular in nature, and may come in a kit having loops 210 and/or retrieval bags 212 in various sizes. In some such versions, the desired sized loop 210 and retrieval bag 212 may be attached to rod 204 prior to insertion of specimen retrieval instrument 200 within a patient. Still various other suitable features, components, configurations, and operabilities that may be incorporated into specimen retrieval instrument 200 will be apparent to those of ordinary skill in the art in view of the teachings herein.

C. Exemplary Telescoping Arm

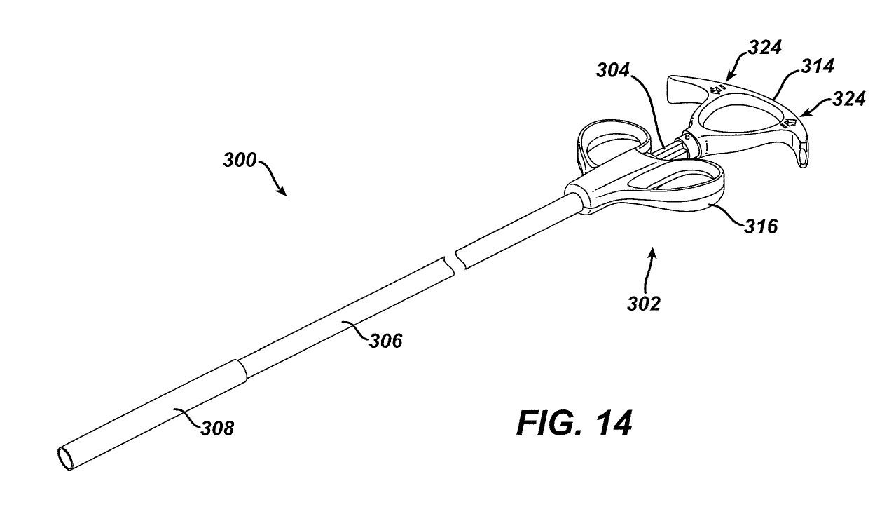

FIGS. 14-17 show another exemplary specimen retrieval instrument 300. Specimen retrieval instrument 300 of the present example includes handle assembly 302, actuating rod 304, introducer tube 306, sheath 308, support arm assembly 342, and retrieval bag 312. These components will be discussed in greater detail below.

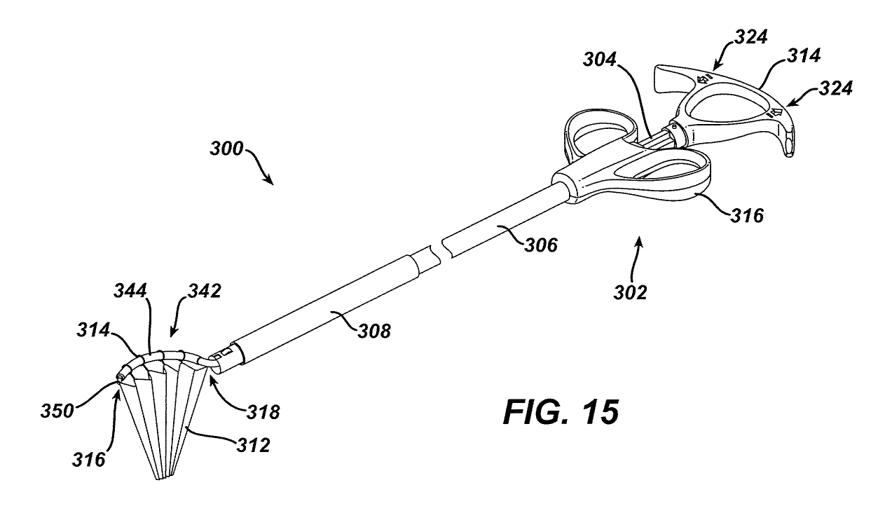

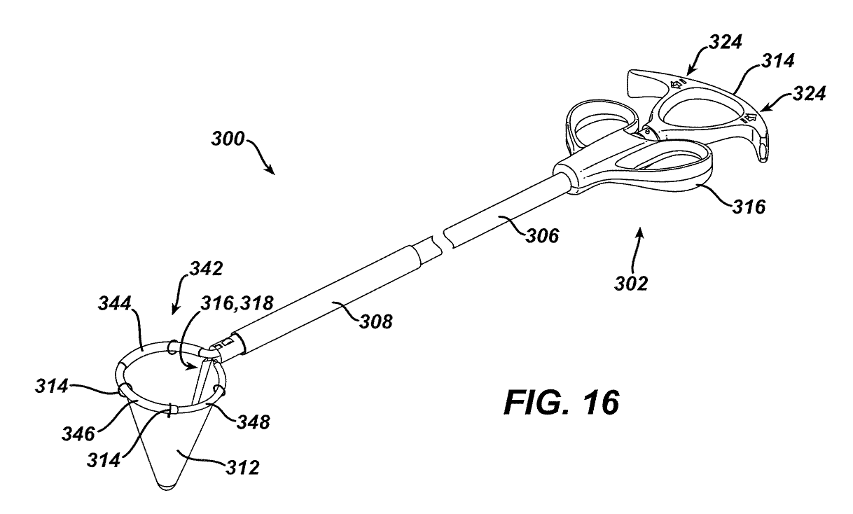

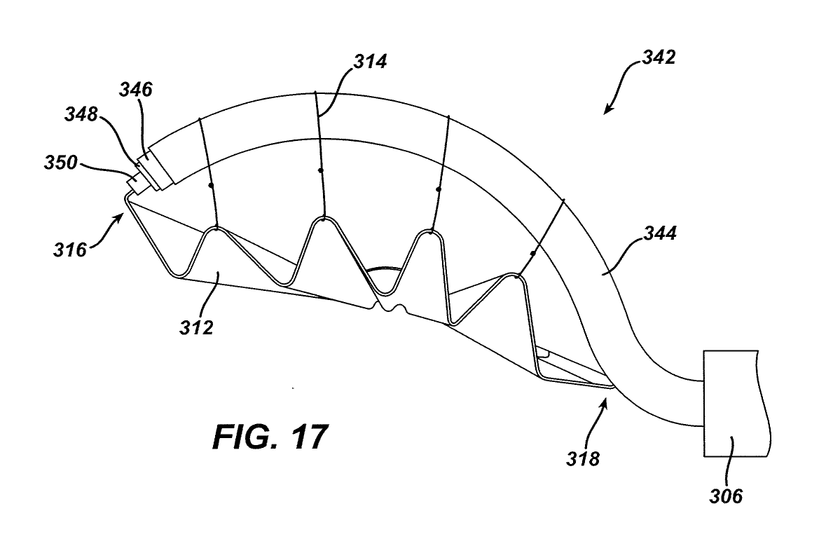

As can be seen from FIGS. 14-15, sheath 308 is configured to translate relative to introducer tube 306. In particular, and as will be described in greater detail below, sheath 308 is translatable from a distal position (FIG. 14) to a proximal position (FIGS. 15-17). Together, introducer tube 306 and sheath 108 are configured to fit within the insertion passageway defined by a device such as a trocar. By way of example only, introducer tube 306 and sheath 308 may present an outer diameter that is between approximately 5 mm (inclusive) and approximately 15 mm (inclusive). Alternatively, these components may have any other suitable dimensions. Support arm assembly 342 carries retrieval bag 312, and is resiliently biased to expand from a compressed configuration (FIG. 14) to an expanded configuration (FIG. 15), and then to an extended configuration (FIGS. 16-17), as will also be described in greater detail below.

FIG. 14 shows specimen retrieval instrument 300 in an undeployed configuration. In this configuration, sheath 308 is in a distal position, substantially enclosing support arm assembly 342 and retrieval bag 312. When specimen retrieval instrument 300 is in the undeployed configuration, specimen retrieval instrument 300 is ready to be inserted within a patient. By way of example only, specimen retrieval instrument 300 may be inserted into a patient via a trocar or other type of access port device, via an incision, via a natural orifice, and/or in any other suitable fashion. Introducer tube 306 has a substantially cylindraceous configuration in the present example. However, it should be understood that introducer tube 306 may have any suitable configuration. In addition, it should be understood that any suitable type of handle assembly may be provided at the proximal end of sheath 308. By way of example only, such a handle assembly may have one or more finger grips, thumb rings, or any other suitable structures, features, or configurations. Such a handle assembly may be configured to facilitate proximal translation of sheath 308 relative to introducer tube 306. In addition or in the alternative, one or more cables, strings, rods, or other features may be operable to provide proximal translation of sheath 308 relative to introducer tube 306. Support arm assembly 342 is resiliently biased to assume a curved configuration as shown in FIG. 15; but support arm assembly 342 is compressible enough in the present example to assume a substantially straight configuration to fit within sheath 308 when sheath 308 is at the distal position shown in FIG. 14.

FIG. 15 shows specimen retrieval instrument 300 in a partially deployed configuration. In this configuration, sheath 308 is in a proximal position, substantially revealing support arm assembly 342 and retrieval bag 312. In the present example, specimen retrieval instrument 300 is transitioned from the undeployed configuration to the deployed configuration after the distal end of specimen retrieval instrument 300 has been inserted within a patient. To transition specimen retrieval instrument 300 from the undeployed configuration to the deployed configuration, sheath 308 is retracted proximally relative to introducer tube 306. It should be understood that such proximal retraction of sheath 308 may be accomplished in a variety of ways. By way of example only, sheath 308 may have a length selected such that a proximal portion of sheath 308 protrudes proximally relative to a trocar or other access port when the distal end of specimen retrieval instrument 300 is inserted in a patient. Thus, such a proximal portion of sheath 308 may be manipulated by a surgeon or other user externally relative to the patient during a surgical procedure. A handle, grip, or other structural feature may be provided at the proximal end of sheath 308 to facilitate such extracorporeal manipulability of sheath 308. In addition or in the alternative, a string, cable, or other feature may be coupled with sheath 308 and may be operable to retract sheath 308 proximally. In addition or in the alternative, a feature that is operable to retract sheath 308 proximally may extend within the interior of introducer tube 306, in addition to or as an alternative to extending along the exterior of introducer tube 306.

In some alternative versions, sheath 308 is removable from introducer tube 306 by pulling sheath 308 off of introducer tube 306 in a distal direction; rather than providing proximal retractability of sheath 308. By way of example only, sheath 308 may be configured like a sleeve or sock, and a separate instrument (e.g., conventional tissue graspers, etc.) may be used to pull sheath 308 distally off of support arm assembly 342 and retrieval bag 312. In addition or in the alternative, sheath 308 may have a perforation or other weakening feature that may be breached to remove sheath 308 by tearing sheath 308 away from introducer tube 306. As yet another merely illustrative alternative, sheath 308 may be formed of an environmentally sensitive material. For instance, sheath 308 may be configured to dissolve or substantially weaken in the presence of bodily fluid, certain temperatures, or other environmental parameters that may be associated with the interior of a patient. Still other various ways in which sheath 308 may be configured and/or operable will be apparent to those of ordinary skill in the art in view of the teachings herein. Furthermore, sheath 308 may even be omitted in some versions.

As shown in FIGS. 14-16, handle assembly 302 includes thumb ring 314, finger rings 316, and passageway (not shown). The passageway extends from the distal end of finger rings 316 to the proximal end of finger rings 316. Actuating rod 304 extends through the passageway, connecting to thumb ring 314 at the proximal end of actuating rod 304. The distal end of actuating rod 304 is coupled with support arm assembly 342. Actuating rod 304 is translatable within introducer tube 306. Finger rings 316 are connected to introducer tube 306. Thumb ring 314 is moveable relative to finger rings 316 and introducer tube 306, and actuating rod 304 moves in unison with thumb ring 314 as thumb ring 314 is moved distally/proximally relative to introducer tube 306. Additional optional features of handle assembly 302 may include markings 324 to indicate directional movement of thumb ring 314 as well as markings (not shown) to indicate the orientation of retrieval bag 312 initially held within sheath 308—e.g. a marking indicating “this end up” such that the opening of retrieval bag 312 is properly located during use.

Referring to FIGS. 15 and 17, specimen retrieval instrument 300 is shown with support arm assembly 342 in a partially deployed position with retrieval bag 312 “closed.” As noted above, support arm assembly 342 is transitioned from an undeployed position (in which support arm assembly 342 is located within sheath 308) to a partially deployed position (in which support arm assembly 342 is revealed by sheath 308) by retracting sheath 308 proximally relative to introducer tube 306 in the present example. In some other versions, support arm assembly 342 is transitioned from the undeployed position to the partially deployed position by advancing actuating rod 304 distally relative to introducer tube 306. In some such versions, sheath 308 may even be omitted, as introducer tube 306 may play a role similar to the role that would otherwise be played by sheath 308. For instance, support arm assembly 342 may be configured to fit within the hollow interior of introducer tube 306 when actuating rod 304 is at a proximal-most position (e.g., a position proximal to the position shown in FIG. 14). Then, actuating rod 304 may be advanced within introducer tube 306 to a first distal position (e.g., the position shown in FIG. 15) by pushing thumb ring 314 distally toward finger rings 316). Actuating rod 304 may then be advanced further to a second distal position (e.g., the position shown in FIG. 16) to fully extend support arm assembly 342 as described below. Of course, support arm assembly 342 may be transitioned from some first position to the partially deployed position shown in FIGS. 15 and 17 in any other suitable fashion.

As noted above, support arm assembly 342 of the present example is resiliently biased to assume an arcuate configuration as shown in FIGS. 15 and 17 once support arm assembly 342 is no longer constrained within sheath 308. Retrieval bag 312 is associated with support arm assembly 342 such that retrieval bag 312 “opens” or fully forms when support arm assembly 342 telescopes to the position shown in FIG. 16. In particular, support arm assembly 342 of the present example comprises a first arm 344, a second arm 346, a third arm 348, and a plug 350. Second arm 346 telescopes within first arm 344. Third arm 348 telescopes within second arm 346, and plug 350 is located at the distal end of third arm 348. When arms 344, 346, 348 are fully extended, support arm assembly 342 assumes a circular shape as shown in FIG. 16. It will also be appreciated by those of ordinary skill in the art, based on the teachings herein, that arms 344, 346, 348 may be configured with internal stops that permit arms 346, 348 to telescopically extend without overextending and becoming separated from one another. With retrieval bag 312 associated with support arm assembly 342, retrieval bag 312 opens as arms 346, 348 telescope, bringing support arm assembly 342 to its fully open configuration.

In the present example, a first end 316 of the top edge of retrieval bag 312 is secured to plug 350; while a second end 318 of the top edge of retrieval bag 312 is secured to a proximal portion of first arm 344. A plurality of loops 314 are secured to the top edge of retrieval bag 312, between ends 316, 318. Loops 314 are further secured to support arm assembly 342. In particular, as shown in FIGS. 15 and 17, first arm 344 is fed through loops 314. As arms 344, 346, 348 telescopingly extend, loops 314 permit the top edge of retrieval bag 312 to follow the arcuate path of telescoping arms 344, 346, 348 like a curtain, such that the top edge of retrieval bag 312 has a generally circular configuration that generally mimics the generally circular configuration defined by extended arms 344, 346, 348 as shown in FIG. 16. While four loops 314 are shown, it should be understood that any other suitable number of loops 314 may be used. In addition, it should be understood that any other suitable structures, components, or features may be used in addition to or in lieu of loops 314. By way of example only, one or more of telescoping arms 344, 346, 348 may be inserted through one or more sleeves, slots, pockets, loops, slits, etc., formed in retrieval bag 312. In addition, the engagement between retrieval bag 312 and support arm assembly 342 is such that ends 316, 318 of retrieval bag 312 substantially overlap each other when arms 344, 346, 348 are fully extended. Such an overlap, along with other components or features that may be included with retrieval bag 312, may provide retrieval bag 312 with a substantially complete container-like configuration when arms 344, 346, 348 are fully extended. In other words, the seam at ends 316, 318 when retrieval bag 312 has the configuration shown in FIG. 16 may be configured to reduce the likelihood of tissue specimens that are inserted in retrieval bag 312 inadvertently falling out of retrieval bag 312 at the seam. Still other suitable ways in which retrieval bag 312 may be configured will be apparent to those of ordinary skill in the art in view of the teachings herein. Similarly, other suitable ways in which retrieval bag 312 may be coupled with support arm assembly 342 will be apparent to those of ordinary skill in the art in view of the teachings herein.

In some versions, the telescoping action of arms 346, 348 may occur using one or more springs or spring-like members positioned within arms 344, 346, 348. The springs or spring-like members may be biased to extend arms 346, 348 when support arm assembly 342 is not constrained by introducer tube 306 or other components. In such versions, as support arm assembly 342 is ejected from within introducer tube 306, springs or spring-like members work to extend arms 346, 348 such that support arm assembly 342 and retrieval bag 312 open.

In some versions, the telescoping action of arms 346, 348 may occur using a push cable or other similar structure that extends through introducer tube 306 or actuating rod 304 and connects with plug 350 of support arm assembly 342. Such a cable may have sufficient tensile strength to allow pushing through the cable yet have sufficient flexibility to permit transverse bending of the cable. Once support arm assembly 342 is ejected from introducer tube 306 by distal advancement of thumb ring 314, the cable may then be advanced distally to cause arms 346, 348 to telescopically extend thereby opening support arm assembly 342 and retrieval bag 312. In the same manner, retracting the cable after extending arms 346, 348 causes arms 346, 348 to telescopically retract thereby closing support arm assembly 342 and retrieval bag 312. In some versions, the cable may further be used to eject support arm assembly 342 from introducer tube 306 instead of distally advancing thumb ring 314.

In some versions, the telescoping action of arms 346, 348 may be controlled through advancement or retraction of thumb ring 314 and associated actuating rod 304. For example, as shown in FIG. 16, thumb ring 314 may be advanced from the proximal position shown in FIG. 15 to cause arms 346, 348 to telescopically extend to open support arm assembly 342 and retrieval bag 312. In such versions, actuating rod 304 may terminate in a cable-like structure, as described above, which connects to plug 350 of support arm assembly 342. The further distal advancement of thumb ring 314 and actuating rod 304 advance the cable-like structure to telescopically extend arms 346, 348. Various other suitable ways in which an actuating rod 304 (or substitute thereof) may be used to actuate support arm assembly 342 will be apparent to those of ordinary skill in the art in view of the teachings herein.

In use, prior to deployment of support arm assembly 342, specimen retrieval instrument 300 may initially have the configuration shown in FIG. 14, where sheath 308 is at a distal position. In this arrangement, the distal portion of specimen retrieval instrument 300 may be inserted within a patient through a suitable incision opening or access port created by a trocar or some other device. Once positioned within the patient, sheath 308 may be retracted proximally. This action reveals support arm assembly 342 and retrieval bag 112. With support arm assembly 342 being so freed from the confines of sheath 308, the resilient bias of support arm assembly 342 causes support arm assembly 342 to assume the arcuate configuration shown in FIGS. 15 and 17. Thumb ring 314 may then be advanced distally toward finger rings 316. This action drives actuating rod 304 distally, causing second arm 346 and third arm 348 to be telescopically extended from first arm 344. Still in other versions, the telescopic extension may occur by use of biased springs or spring-like members associated with arms 344, 346, 348. Regardless of the mode for telescopically extending arms 346 348 of support arm assembly 342, such extension causes formation of an opened associated retrieval bag 312 in the present example. Once retrieval bag 312 has been formed/opened, specimen retrieval instrument 300 may have the arrangement shown in FIG. 16. One or more tissue specimens, etc., may then be placed in retrieval bag 312.

In some versions, ends 316, 318 are configured such that they are substantially secured to each other after support arm assembly 342 reaches the configuration shown in FIG. 16. For instance, an adhesive and/or interlocking features may be provided at ends 316, 318 to substantially secure ends 316, 318 to each other after support arm assembly 342 reaches the configuration shown in FIG. 16. In some such versions, retrieval bag 312 is released from support arm assembly 342 when arms 346, 348 are retracted after having been extended to the configuration shown in FIG. 16. For instance, such retraction of arms 346, 348 may be provided by retracting thumb ring 314 proximally relative to finger grips 316, which may in turn retract actuating rod 304 proximally relative to introducer tube 306. Alternatively, retrieval bag 312 may be removed from support arm assembly 342 in any other suitable fashion. Regardless of how or when retrieval bag 312 is removed from support arm assembly 342, sheath 308 may be advanced distally over support arm assembly 342 after arms 346, 348 have been retracted to the position shown in FIGS. 15 and 17. Such distal advancement of sheath 308 may substantially straighten support arm assembly 342, allowing sheath 308 to at least substantially envelop support arm assembly 342 for removal of specimen retrieval instrument 300 from the patient.

In some versions, specimen retrieval instrument 300 is configured such that retrieval bag 312 may be removed from specimen retrieval instrument 300 while retrieval bag 312 is within the patient. Some such versions facilitate removal of retrieval bag 312 separate from removal of the other components of specimen retrieval instrument 300. In some versions, this may be accomplished by, among other ways, retrieval bag 312 being removable from loop support arm assembly 342 as noted above. Furthermore, in some versions specimen retrieval instrument 300 may include a closure string connected to retrieval bag 312 and having a slipknot attachment to actuating rod 304. Pulling the slipknot loose and retracting introducer tube 306 may permit detachment of retrieval bag 312 and the closure string from the other components of specimen retrieval instrument 300. In some such versions, a user may pull the closure string to close retrieval bag 312. By way of example only, such a closure mechanism may be configured in accordance with the teachings of U.S. Pat. No. 6,409,733, entitled “Specimen Retrieval Bag,” issued Jun. 25, 2002, which is incorporated herein by reference. Still in other versions, it may be feasible to incorporate a closure string with retrieval bag 312, and to release retrieval bag 312 from specimen retrieval instrument 300 such that retrieval bag 312 may be removed from the patient separate from other components of specimen retrieval instrument 300.

While the above description provides adequate disclosure to enable one of ordinary skill in the art to make and use specimen retrieval instrument 300, based on the teachings herein, those of ordinary skill in the art will appreciate that various other modifications may provide additional features or functionality. For example, in some versions modifications may include actuating rod 304 and/or sheath 308 comprising features operable with features of introducer tube 306 or other components to prevent inadvertent retraction of sheath 308 (and/or inadvertent extension of actuating rod 304) and premature deployment of support arm assembly 342. For example, sheath 308 and/or actuating rod 304 may include a lock or stop that may be released once a user is ready to deploy support arm assembly 342. Still in other versions, modifications may include actuating rod 304 comprising features operable with features of introducer tube 306 or other components to prevent inadvertent retraction of actuating rod 304 after initial deployment of support arm assembly 342. For example, actuating rod 304 may be associated with a resilient tab configured to engage a corresponding opening in a sidewall of introducer tube 306 upon deployment of support arm assembly 342. The engagement between the resilient tab and the opening in the sidewall of introducer tube 306 may act as a locking mechanism that prevents inadvertent retraction of actuating rod 304 and therefore inadvertent premature retraction of support arm assembly 342. Other ways in which inadvertent retraction and/or advancement of sheath 308 and/or actuating rod 304 may be avoided through various features of specimen retrieval instrument 300 will be apparent to those of ordinary skill in the art in view of the teachings herein.

In some versions, modifications may include providing support arm assembly 342 with various lengths of telescopic arms such that support arm assembly 342 is provided in a variety of sizes that may be further paired with various sized retrieval bags 312. In such versions, the desired size support arm assembly 342 and retrieval bag 312 may be attached to actuating rod 304 prior to insertion of specimen retrieval instrument 300 within a patient. Still various other suitable features, components, configurations, and operabilities that may be incorporated into specimen retrieval instrument 300 will be apparent to those of ordinary skill in the art in view of the teachings herein.

II. Exemplary Retrieval Bag Opening Assistance

Another area to consider when designing a specimen retrieval instrument is the modes used to facilitate opening of a retrieval bag once the bag has been deployed from the specimen retrieval instrument. The following section will discuss several exemplary retrieval bag assistance modes, which may include use of a spiral reinforced rib retrieval bag, an unrolling stay, and a twist actuation. Based on the teachings herein, other deployment modes will be apparent to those of ordinary skill in the art.

A. Exemplary Spiral Reinforced Rib Retrieval Bag

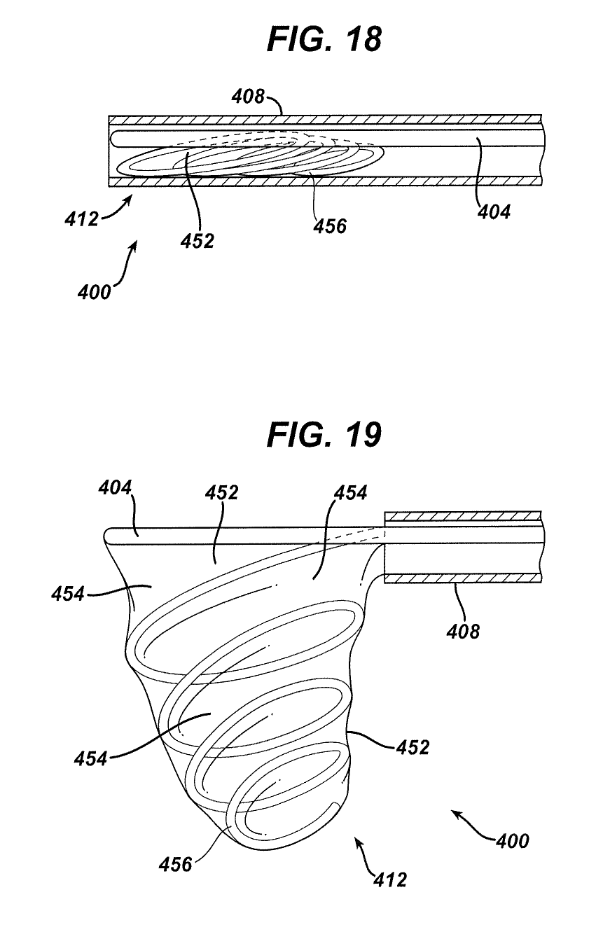

FIGS. 18-19 show the distal portion 400 of a specimen retrieval instrument incorporating spiral reinforced rib retrieval bag 412. Retrieval bag 412 is collapsible to fit within introducer tube 408 or some other component of distal portion 400 of the specimen retrieval instrument. As shown in FIG. 19, retrieval bag 412 may be deployed from within introducer tube 408, and upon deployment, retrieval bag 412 resiliently opens for receipt of a specimen.

Retrieval bag 412 of the present example comprises a membrane 452, a plurality of ribs 454 associated with membrane 452, and a spiral member 456. Retrieval bag 412 is secured to the distal end of an actuating rod 404 such that translation of actuating rod 404 may cause retrieval bag 412 to translate in unison with actuating rod 404. In the present example, actuating rod 404 is translatable from a proximal position within introducer tube 408 (FIG. 18) to a distal position relative to introducer tube (FIG. 19). In some other versions, actuating rod 404 may be substantially stationary, with translation of introducer tube 408 causing deployment of retrieval bag 412. Actuating rod 404 may be attached to both membrane 452 and spiral member 456 as shown in FIG. 19. Ribs 454 may extend vertically along membrane 452. In some versions ribs 454 may comprise sections of reinforced or thicker material of membrane 452. In some other versions ribs 454 may comprise sections of membrane 452 that are reinforced by stitching or embossing. Ribs 454 may contribute to providing retrieval bag 412 with directionality that is consistent with retrieval bag 412 assuming an open position. At the same time, ribs 454 are not overly restrictive to inhibit retrieval bag 412 from collapsing to fit within introducer tube 408. In some other versions, ribs 454 are simply omitted altogether.

Spiral member 456 is collapsible to fit within introducer tube 408 or some other component of distal portion 400 of the specimen retrieval instrument. Spiral member 456 is biased such that the looping portions that comprise spiral member 456 have a diameter consistent with the diameter of the retrieval bag opening defined by membrane 452. In particular, top portions of spiral member 456 define diameters that are greater than diameters defined by bottom portions of spiral member 456, such that the diameter defined by spiral member 456 gets progressively smaller from the top of spiral member 456 toward the bottom of spiral member 456. It should be understood that spiral member 456 may compress in various ways. For instance, spiral member 456 may compress along an axis that is vertically transverse to the longitudinal axes defined by actuating rod 404 and introducer tube 408. In other words, spiral member 456 may compress along an axis that is vertical in the views shown in FIGS. 18-19. In addition, spiral member 456 of the present example compresses along a plane that is laterally transverse to the longitudinal axes defined by actuating rod 404 and introducer tube 408. In addition or in the alternative, spiral member 456 may compress in any other suitable fashion. Spiral member 456 is resiliently biased to assume the extended configuration shown in FIG. 19. As with any other resilient component mentioned herein, spiral member 456 may be formed of any suitable material or combination of materials, including but not limited to metal (e.g., stainless steel, nitinol, steel spring alloys, copper spring alloys, etc.), plastic, and/or metal reinforced plastic.

In use, once distal portion 400 of specimen retrieval instrument is positioned within a patient, retrieval bag 412 may be deployed from within introducer tube 408 by any suitable means, e.g. extending actuator rod 404 distally relative to the patient or retracting introducer tube 408 proximally relative to the patient. After deployment, spiral member 456 releases from its constrained position forcing retrieval bag 412 to its fully open position. Working separately from, or in conjunction with, spiral member 456, ribs 454 also operate to assist retrieval bag 412 in reaching the fully open position. After a specimen has been collected within retrieval bag 412, retrieval bag 412 may be closed and subsequently removed from within the patient by any suitable means described previously.

While the above description provides adequate disclosure to enable one of ordinary skill in the art to make and use a specimen retrieval instrument incorporating a spiral rib reinforced retrieval bag 412, based on the teachings herein, those of ordinary skill in the art will appreciate that various other modifications may provide additional features or functionality. For example, retrieval bag 412 may be adapted to fit with or work with other types of specimen retrieval instruments, not just that shown in FIGS. 18 and 19. Still various other suitable features, components, configurations, and operabilities that may be incorporated into retrieval bag 412 and any associated specimen retrieval instrument will be apparent to those of ordinary skill in the art in view of the teachings herein.

B. Exemplary Unrolling Stay

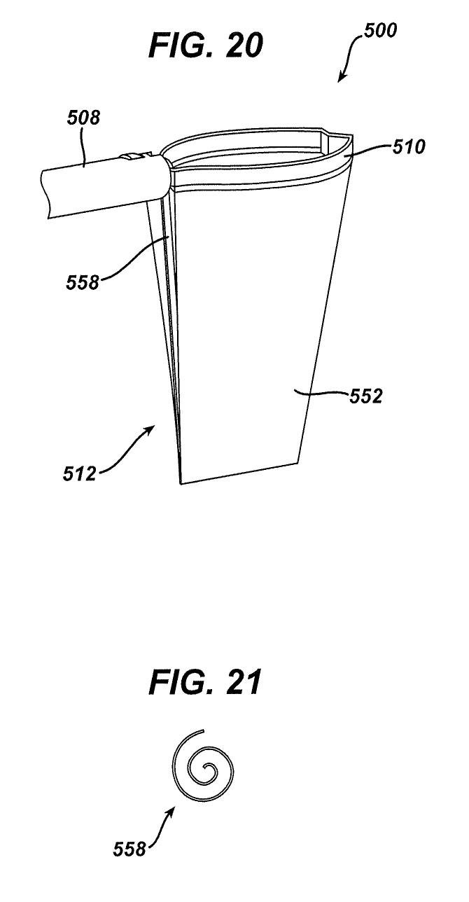

FIG. 20 shows the distal portion 500 of a specimen retrieval instrument incorporating a resilient unrolling stay 558 into retrieval bag 512. In addition to stay 558, retrieval bag 512 may comprise a membrane 552. Retrieval bag 512 is secured to a resilient loop 510 that extends distally from an introducer tube 508. In particular, membrane 552 may removably attach to loop 510. Loop 510 may be associated with an actuating rod or some other component, such that loop 510 may be selectively deployed from within introducer tube 508 as described previously. In a pre-deployment position, loop 510 is sufficiently flexible to straighten somewhat such that loop 510 fits with retrieval bag 512 within introducer tube 508. Loop 510 may further be resiliently biased to assume a circular or elliptical shape upon deployment from within introducer tube 508, as also described previously. Accordingly, the specimen retrieval instrument whose distal portion 500 is shown in FIG. 20 may be transitioned from an undeployed configuration (in which loop 510, retrieval bag 512 and stay 558 are all located within introducer tube 508) to a deployed (in which loop 510, retrieval bag 512 and stay 558 are extended from the distal end of introducer tube 508 and are exposed relative to introducer tube 508).

In addition to loop 510 being adaptable to fit within introducer tube 508, retrieval bag 512 is collapsible to fit within introducer tube 508 as well. Stay 558 is secured to membrane 552, such as by being adhered to membrane 552 and/or being inserted in a pocket of membrane 552, etc. As shown in FIG. 21, stay 558 may be in a rolled up position when retrieval bag 512 and loop 510 are in pre-deployment positions. Stay 558 may be formed of a semi-rigid material, such as a plastic, metal, metal-reinforced plastic, and/or any other suitable material or combination of materials. When deployed from introducer tube 508, stay 558 is resiliently biased to assume a substantially straight configuration, such that stay 558 will naturally and automatically unroll. With stay 558 in communication with membrane 552 of retrieval bag 512, unrolling of stay 558 assists membrane 552 of retrieval bag 512 in attaining an open and unrolled position. With stay 558 unrolled and retrieval bag 512 open, a specimen may now be placed within retrieval bag 512.

In use, once distal portion 500 of specimen retrieval instrument is positioned within a patient, retrieval bag 512 may be deployed from within introducer tube 508 by any suitable means, e.g. extending an actuator rod distally or retracting introducer tube 508 proximally, etc. After deployment, stay 558 releases from its constrained rolled-up position forcing retrieval bag 512 to its unfurled open configuration. A resilient bias of loop 510 may also assist in opening retrieval bag 512. After a specimen has been collected within retrieval bag 512, retrieval bag 512 may be closed by retracting loop 510, or pulling a closure string as discussed previously. Retrieval bag 512 may then be removed from within the patient by any suitable means described previously or otherwise.

While the above description provides adequate disclosure to enable one of ordinary skill in the art to make and use a specimen retrieval instrument incorporating unrolling stay 558 in retrieval bag 512, based on the teachings herein, those of ordinary skill in the art will appreciate that various other modifications may provide additional features or functionality. For example, retrieval bag 512 may be adapted to fit with or work with other types of specimen retrieval instruments, not just that shown in FIG. 20. Still various other suitable features, components, configurations, and operabilities that may be incorporated into retrieval bag 512 and any associated specimen retrieval instrument will be apparent to those of ordinary skill in the art in view of the teachings herein.

C. Exemplary Twist Actuation

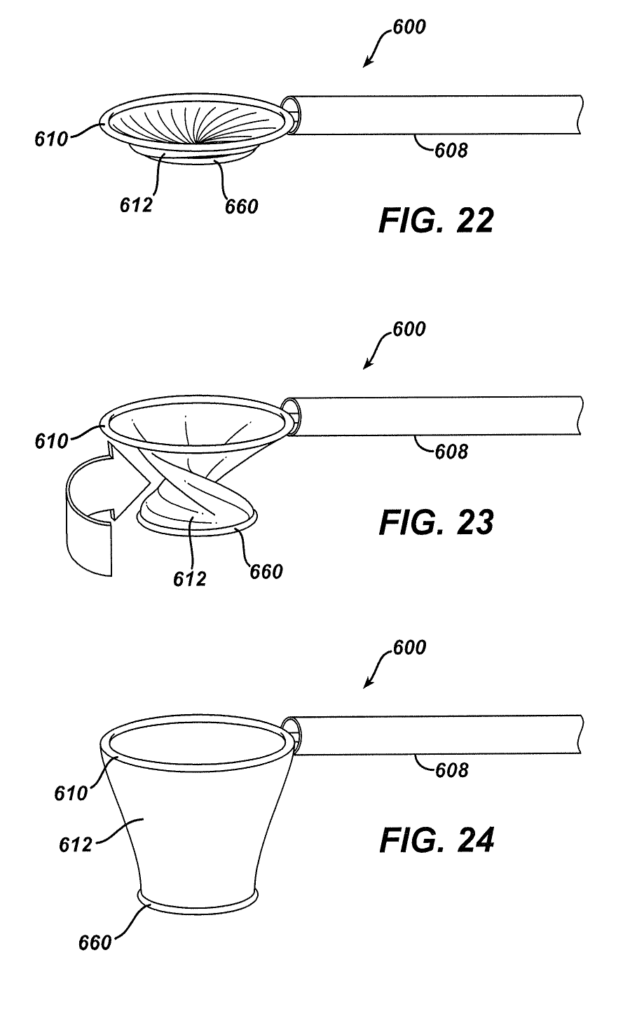

FIGS. 22-24 show the distal portion 600 of specimen retrieval instrument incorporating dual loop twisting retrieval bag 612. Distal portion 600 of specimen retrieval instrument may include introducer tube 608, an actuating rod (not shown), first loop 610, second loop 660, and retrieval bag 612. First loop 610 and second loop 660 are each resiliently biased to assume a substantially circular configuration. However, first loop 610 and second loop 660 are each sufficiently flexible to compressingly fit within introducer tube 608. In addition, first loop 610 is secured to the open top end of retrieval bag 612; while second loop 660 is secured to the closed bottom end of retrieval bag 612. Prior to being captured within introducer tube 608, second loop 660 may be rotated relative to first loop 610, thereby twisting retrieval bag 612. Second loop 660 may then be placed adjacent to first loop 610 and/or within first loop 610, with loops 610, 660 and twisted retrieval bag 612 then being positioned within introducer tube 608.

Prior to the position shown in FIG. 22, the specimen retrieval instrument may be configured in an initial position where first loop 610, second loop 660, and retrieval bag 612 are constrained within introducer tube 608. After any suitable deployment mode has been exercised, as described above or otherwise (e.g., retraction of sheath, advancement of rod, etc.), first loop 610, second loop 660, and retrieval bag 612 are exposed distally relative to introducer tube 608 and are no longer constrained by introducer tube 608, as shown in FIG. 22. As described above previously, first loop 610 may incorporate a resilient bias such that first loop 610 assumes a circular shape once deployed relative to introducer tube 608. Second loop 660 may also incorporate a similar resilient bias. As shown in FIG. 23, second loop 660 may be rotated relative to first loop 610, such that retrieval bag 612 untwists. In particular, rotation of second loop 660 relative to first loop 610 causes retrieval bag 612 to unwind to its fully open position. As shown in FIG. 24, with retrieval bag 612 fully open, retrieval bag 612 is prepared for receipt of a specimen.

Rotation of second loop 660 may to transition from the configuration shown in FIG. 22 to the configuration shown in FIG. 24 may be accomplished in a variety of ways. For example, in some versions second loop 660 is rotated by using a separate instrument such as a grasper. Still in other versions, first loop 610 may be rotated relative to second loop 660 by first loop 610 being associated with pull cables connected to opposing sides of first loop 610. Pull cables may pass through introducer tube 608, extending outside the patient to a handle assembly area of the specimen retrieval instrument. In the present example, however, first loop 610 does not rotate, and is simply fixedly secured to a rod that is positioned within introducer tube 608. Also in the present example, tension and/or a resilient bias of retrieval bag 612 urges retrieval bag 612 to “unwind” as soon as it is freed from the confines of introducer tube 608 or an external sheath, such that retrieval bag 612 transitions from the configuration shown in FIG. 22 to the configuration shown in FIG. 24 substantially on its own. Still other suitable ways in which retrieval bag 612 may be deployed and transition from the configuration shown in FIG. 22 to the configuration shown in FIG. 24 will be apparent to those of ordinary skill in the art in view of the teachings herein.

Once a specimen has been captured within retrieval bag 612, first loop 610 may be retracted proximally to contact introducer tube 608. Such contact with introducer tube 608 may overcome the resilient bias of first loop 610, thereby causing first loop 610 to close. This action similarly may close retrieval bag. In addition or in the alternative, and as described above, retrieval bag 612 may be closed through the use of a closure string. In any event, once a specimen has been captured within retrieval bag 612, retrieval bag 612 and other components of the specimen retrieval instrument may be removed from the patent by any suitable means as described previously or otherwise.

III. Exemplary Alternative Instrument

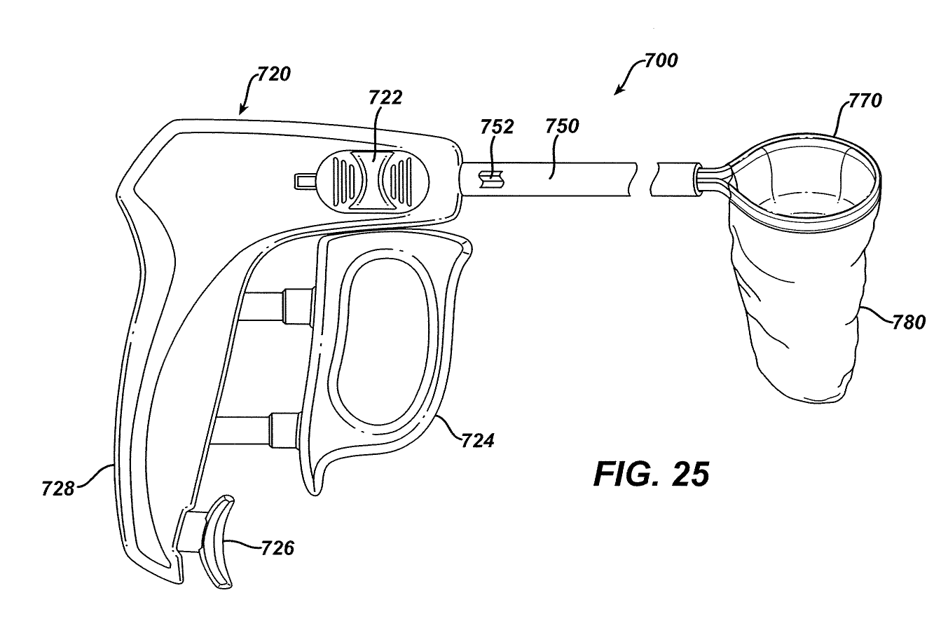

FIG. 25 depicts a merely illustrative alternative specimen retrieval instrument 700. Specimen retrieval instrument 700 of this example includes a handle portion 720, an elongate shaft 750, a resilient hoop 770, and a retrieval bag 780. Elongate shaft 750 extends distally from handle portion 720. An actuating rod 752 extends through elongate shaft 750 and is translatable within shaft 750. In particular, a proximal end of actuating rod 752 is coupled with components of handle portion 720 as will be described in greater detail below; while the distal end of actuating rod 752 is coupled with resilient hoop 770. Actuating rod 752 is thus operable to selectively deploy resilient hoop 770 from a retracted position (in which resilient hoop 770 is at least substantially positioned within elongate shaft 750) to an extended position (in which resilient hoop 770 is exposed and positioned distal relative to elongate shaft 750 as shown in FIG. 25). Shaft 750 may have an outer diameter that is between approximately 5 mm (inclusive) and approximately 15 mm (inclusive). Alternatively, these components may have any other suitable dimensions. Alternatively, shaft 750 may have any other suitable outer diameter.

Retrieval bag 780 is secured to resilient hoop 770, such that retrieval bag 780 is retracted or deployed with actuating rod 752 relative to shaft 750. In other words, retrieval bag 780 is configured to fit with resilient hoop 770 within shaft 750. Retrieval bag 780 may be folded, rolled, wadded up, or otherwise manipulated to fit within shaft 750. Furthermore, retrieval bag 780 may be configured in accordance with the teachings relating to any retrieval bag mentioned herein; or retrieval bag 780 may have any other suitable configuration. Resilient hoop 770 is resiliently biased to assume the expanded, generally circular configuration shown in FIG. 25. However, as noted above, resilient hoop 770 may still be compressed to fit within shaft 750. Of course, resilient hoop 770 may have any other suitable configuration. By way of example only, resilient hoop 770 may instead comprise two or more arms rather than a hoop.

Handle portion 720 includes a slider 722, a cinching trigger 724, and a locking trigger 726. Slider 722 is coupled with the proximal end of actuating rod 752, and is operable to translate actuating rod 752 longitudinally within shaft 750. In particular, slider 722 may be slid distally to advance resilient hoop 770 and thereby deploy resilient hoop 770 and retrieval bag 780 from within shaft 750. Of course, slider 722 may be modified, substituted, or supplemented in a variety of ways (or even be omitted) as will be apparent to those of ordinary skill in the art in view of the teachings herein. Cinching trigger 724 is coupled with a closure string (not shown), which is fed through retrieval bag 780 to selectively cinch retrieval bag 780 closed. In particular, cinching trigger 724 is operable to cinch retrieval bag 780 closed by squeezing cinching trigger 724 toward pistol grip 728 of handle portion 720. Cinching trigger 724 is resiliently biased away from pistol grip 728 in the present example, such that an operator will be squeezing cinching against the resilient bias of a spring or other component when the operator is cinching retrieval bag 780. In some versions, locking trigger 726 is operable to lock a cinched configuration of retrieval bag 780. For instance, in some versions, the operator will squeeze cinching trigger 724 until a desired cinching position is obtained, and then the operator will actuate locking trigger 726 to substantially secure the selected cinching position, such that the locking trigger 726 prevents the resilient bias of cinching trigger 724 from un-cinching retrieval bag 780 when cinching trigger 724 is released. As another merely illustrative variation, a ratcheting mechanism may substantially lock the cinching position of cinching trigger 724 and retrieval bag 780; and locking trigger 726 may release the ratcheting mechanism to permit adjustment of a cinched position. Like slider 722, triggers 724, 726 may be modified, substituted, or supplemented in a variety of ways (or even be omitted) as will be apparent to those of ordinary skill in the art in view of the teachings herein.

In use, the distal end of shaft 750 may be introduced to a surgical site via a trocar or other device, etc. Slider 722 may be advanced distally to deploy resilient hoop 770 and retrieval bag 780 within the patient. A specimen may then be placed within retrieval bag 780. Next, cinching trigger 724 may be actuated to cinch retrieval bag 780; and locking trigger 726 may be actuated to lock a cinched position. The distal end of specimen retrieval instrument 700 may then be removed from the patient substantially simultaneously with the trocar or other access port device. Alternatively, specimen retrieval instrument 700 may be removed from the patient, in whole or in pieces, separate from removal of the trocar from the patient. In addition or in the alternative, some versions of specimen retrieval instrument 700 may permit removal of retrieval bag 780 from specimen retrieval instrument 700 before anything is withdrawn from the patient. In some such versions, portions of specimen retrieval instrument 700 may be removed from the patient separately from and before removal of retrieval bag 780 from the patient. If desired, an integral string or other feature may be used to remove retrieval bag 780 from the patient after at least part of specimen retrieval instrument has been removed from the patient. Still various other suitable features, components, configurations, and operabilities that may be incorporated into specimen retrieval instrument 700 will be apparent to those of ordinary skill in the art in view of the teachings herein.

As noted above, the various versions of specimen retrieval instruments described herein, including but not limited to the various versions of retrieval bags described herein, may be used in a conventional endoscopic procedure that includes the insertion of the introducer tube or other component through a small opening, e.g., an incision, natural orifice, or trocar access port, etc. Of course, specimen retrieval instruments described herein may be used in conjunction with any other suitable surgical or medical procedure, such as endoscopic/laparoscopic procedures, open surgical procedures, or robotic-assisted surgery, etc. Still other various settings and combinations in which specimen retrieval instruments described herein may be used will be apparent to those of ordinary skill in the art in view of the teachings herein.

While several specimen retrieval instruments, and components thereof, have been discussed in detail above, it should be understood that the components, features, configurations, and methods of using the specimen retrieval instruments discussed are not limited to the contexts provided above. In particular, components, features, configurations, and methods of use described in the context of one of the specimen retrieval instruments may be incorporated into any of the other specimen retrieval instruments. One merely exemplary additional feature that may be provided in any of the specimen retrieval instruments described herein includes retrieval bags having various sizes and geometries. For example, some specimen retrieval instruments may be designed with small, medium, or large retrieval bags. It should also be understood that any of the specimen retrieval instruments and tissue retrieval bags described herein may be capable of receiving tissue specimens and removing tissue specimens from a patient without such tissue specimens needing to be morcellated or otherwise reduced in size before being received and removed by the specimen retrieval instrument and bag. Still other additional and alternative suitable components, features, configurations, and methods of using the specimen retrieval instruments will be apparent to those of ordinary skill in the art in view of the teachings herein.

Other features and modifications that will be appreciated based on the teachings herein involve methods of attaching a retrieval bag to any of the various arms and loops or other components of a specimen retrieval instrument described above. For example, retrieval bags may be configured with one or more sleeves, slots, pockets, loops, slits, etc., for receiving any of the various arms and loops described above. In other versions, retrieval bags may be connected to any of the various arms, loops, or other components using suitable mechanical or chemical means. It will further be appreciated that in some versions the retrieval bag may be detachable from the other components of the specimen retrieval instrument, while in some other versions the retrieval bag may be inseparable from the specimen retrieval instrument. Still other additional and alternative suitable components, features, configurations, and methods of attaching retrieval bags with the other components of a specimen retrieval instrument will be apparent to those of ordinary skill in the art in view of the teachings herein.

While several retrieval bags and deployment mechanisms have been discussed in detail above, it should be understood that the components, features, configurations, and methods of using the bags and deployment mechanisms discussed are not limited to the contexts provided above. In particular, components, features, configurations, and methods of use described in the context of one of the retrieval bags may be incorporated into any of the other retrieval bags. One merely exemplary additional feature that may be provided in any of the retrieval bags described herein is one or more weld lines. Such weld lines may be intermittent or continuous along the length of the bag. Such weld lines, offering alternating areas of stiffness along the surface of the bag, may enhance the closure of a bag due to the tendency of areas of lesser stiffness to buckle, deform, or fold. In this way, a retrieval bag may be forced or encouraged to buckle or fold in a desired manner as the bag is closed. Still other additional and alternative suitable components, features, configurations, and methods of using the above-described retrieval devices will be apparent to those of ordinary skill in the art in view of the teachings herein.

While the tissue retrieval instruments of the above-described examples are actuated manually by advancing a thumb ring distally relative to finger rings, retracting a sheath relative to an introducer tube, advancing a slider, or in some other manual fashion, etc., it should be understood that any of the tissue retrieval instruments described herein may instead be actuated in any other suitable fashion. By way of example only, a tissue retrieval instrument may instead be actuated electromechanically (e.g., using one or more electrical motors, solenoids, etc.), pneumatically, and/or hydraulically. Various suitable ways in which such alternative forms of actuation may be provided in a tissue retrieval instrument will be apparent to those of ordinary skill in the art in view of the teachings herein. Similarly, various other suitable ways in which a tissue retrieval instrument may be actuated will be apparent to those of ordinary skill in the art in view of the teachings herein.

It should be understood that any of the retrieval bags described herein may have various types of construction. By way of example only, any of the retrieval bags described herein may be constructed from at least one layer of an elastomeric or polymeric material such as but not limited to polyurethane, polyethylene, polypropelene, polyester (Dura-lar), Poly-isoprene, silicone, vinyl, or a polytetrafluroethyelene (Teflon®). For example, any retrieval bag described herein may comprise a single layer of elastomeric or polymeric material. Alternatively, any retrieval bag described herein may be formed of two or more layers of material. For instance, two or more layers of a retrieval bag wall may be aligned and joined together by adhesives, heat welding, heat staking, RF welding, ultrasonically welding, or other suitable method of attachment. Any retrieval bag described herein may also be cut at an angle to provide a taper or special shapes suitable for specific organs of body (e.g., tissue shapes, etc.), which may facilitate removal of the retrieval bag from a patient. Furthermore, any retrieval bag described herein may incorporate flexible metal meshes, thermoformed plastic meshes, fabrics, or aramid fibers such as Kevlar® for reinforcement. Still other suitable materials that may be used to form retrieval bags as described herein, including combinations of materials, will be apparent to those of ordinary skill in the art in view of the teachings herein. Similarly, various other suitable compositions of the walls of the retrieval bags described herein, including but not limited to various structures, components, and features that may be incorporated into the walls of the retrieval bags described herein, will be apparent to those of ordinary skill in the art in view of the teachings herein.

In any of the above described tissue retrieval instruments, the tissue retrieval bag may include a fold-over flap (not shown) for closing the bag. For instance, such a fold-over flap may be used instead of (or in addition to) using a string to effect closure of the bag. Such a fold-over flap may include an adhesive (e.g., pressure sensitive adhesive, etc.) that substantially keeps the flap in a closed position after the flap has been moved to a closed position. A peel-away strip or similar feature may be used to cover such an adhesive before the flap is closed. A conventional grasping instrument or other type of device may be used to peel the peel-away strip and/or close the flap over the mouth of the bag while the bag is still inside the patient. In some other variations, a tissue retrieval bag may be formed at least in part of a material that provides significant static adhesion or other type of adhesion to itself. For instance, the interior surfaces of the tissue retrieval bag may be configured to adhere to each other and/or to adhere to tissue/objects placed in the bag, to reduce the likelihood of tissue/objects in the bag falling out of the bag. In some such versions, a closure string is omitted. Other suitable variations of a tissue retrieval bag will be apparent to those of ordinary skill in the art in view of the teachings herein.

Versions of the devices disclosed herein have application in conventional endoscopic and open surgical instrumentation as well as application in robotic-assisted surgery.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, embodiments of the device may be disassembled, and any number of the particular pieces or parts of the device may be selectively replaced or removed in any combination. Upon cleaning and/or replacement of particular parts, embodiments of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various versions in the present disclosure, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

What is claimed is:

1. A surgical instrument for removal of material from a patient, the surgical instrument being operable to transition from an undeployed configuration to a deployed configuration, wherein the surgical instrument comprises:
a. a tubular member, the tubular member having a proximal end and a distal end, the tubular member further defining an inner diameter;
b. a resilient member associated with the tubular member, wherein the resilient member is biased to assume an expanded configuration, wherein the resilient member is collapsible to a compressed configuration, wherein the resilient member fits within the inner diameter of the tubular member when the resilient member is in the compressed configuration; and
c. a retrieval bag secured to the resilient member, wherein the retrieval bag is configured to receive a tissue specimen when the resilient member is in the expanded configuration.

2. The surgical instrument of claim 1, wherein the tubular member defines a longitudinal axis, wherein the resilient member is twisted about the longitudinal axis when the tubular resilient member is in the compressed configuration within the tubular member.

3. The surgical instrument of claim 1, wherein the resilient member has a loop shape.

4. The surgical instrument of claim 3, further comprising a rod having a proximal end and a distal end, wherein the resilient member is secured to the distal end of the rod, wherein the tubular member is translatable relative to the rod.

5. The surgical instrument of claim 4, wherein the resilient member is rotatable about the distal end of the rod to transition from a retracted position to an extended position, wherein the resilient member is in the expanded configuration when the resilient member is at the extended position.

6. The surgical instrument of claim 5, wherein the rod defines a transversely oriented recess configured to retain at least a portion of the resilient member between the rod and the inner diameter of the tubular member when the resilient member is rotated to the retracted position and contained within the tubular member.

7. The surgical instrument of claim 1, wherein the resilient member comprises a resilient stay coupled with the retrieval bag, wherein the resilient stay is resiliently biased to assume a substantially straight configuration that is transverse to a longitudinal axis defined by the tubular member when the resilient member is in the expanded configuration.

8. The surgical instrument of claim 7, wherein the resilient stay is configured to roll into a spiral configuration when the resilient stay is in the compressed configuration.

9. The surgical instrument of claim 1, wherein the resilient member comprises a spiral spring coupled with the retrieval bag, wherein spiral spring has a spiral shape that encircles an axis that is transverse to a longitudinal axis defined by the tubular member.

10. The surgical instrument of claim 1, wherein the resilient member comprises telescoping segments.

11. The surgical instrument of claim 10, wherein the telescoping segments are resiliently biased to assume a generally arcuate configuration, wherein the telescoping segments are extendable relative to each other to form a generally circular configuration.

12. The surgical instrument of claim 10, wherein the telescoping segments are resiliently biased to extend relative to each other from a collapsed configuration.

13. The surgical instrument of claim 1, wherein the resilient member comprises a first resilient hoop secured to a top portion of the retrieval bag, the surgical instrument further comprising a second resilient hoop secured to a bottom portion of the retrieval bag.

14. The surgical instrument of claim 13, wherein the retrieval bag is twistable to rotate the second hoop relative to the first hoop.

15. The surgical instrument of claim 1, further comprising an outer sheath translatable relative to the tubular member, wherein the outer sheath is positionable over the resilient member to constrain the resilient member in the compressed configuration, wherein the sheath is movable relative to the resilient member to allow the resilient member to transition to the expanded configuration.

16. The surgical instrument of claim 1, further comprising an actuating rod, wherein the resilient member is coupled with the actuating rod, wherein the actuating rod is operable to actuate the resilient member to at least partially transition the resilient member to the expanded configuration.

17. The surgical instrument of claim 16, wherein the actuating rod is translatable relative to the tubular member, wherein the actuating rod is operable to advance the resilient member from a proximal position in which the tubular member constrains the resilient member in the compressed configuration to a distal position in which the resilient member is exposed relative to the tubular member to transition to the expanded configuration.

18. The surgical instrument of claim 16, further comprising a handle assembly, wherein the handle assembly comprises a thumb ring and a pair of finger loops, wherein the thumb ring is operable to translate the actuating rod within the introducer tube.

19. A surgical instrument for removal of material from a patient, the surgical instrument being operable to transition from an undeployed configuration to a deployed configuration, wherein the surgical instrument comprises:
a. a tubular member, the tubular member having a proximal end and a distal end, the tubular member further defining an inner diameter;
b. a resilient hoop associated with the tubular member, wherein the resilient hoop is biased to assume an expanded configuration, wherein the resilient hoop is collapsible to a compressed configuration, wherein the resilient hoop fits within the inner diameter of the tubular member when the resilient hoop is in the compressed configuration; and
c. a retrieval bag having an open top portion secured to the resilient hoop, wherein the retrieval bag is configured to receive a tissue specimen when the resilient hoop is in the expanded configuration.

20. A surgical instrument for removal of material from a patient, the surgical instrument being operable to transition from an undeployed configuration to a deployed configuration, wherein the surgical instrument comprises:
a. a tubular member, the tubular member having a proximal end and a distal end, the tubular member further defining an inner diameter, wherein the tubular member is sized to fit in a trocar port;
b. an expandable member associated with the tubular member, wherein the expandable member is biased to assume an expanded configuration, wherein the expandable member is collapsible to a compressed configuration, wherein the expandable member fits within the inner diameter of the tubular member when the expandable member is in the compressed configuration; and
c. a retrieval bag having an open top portion secured to the expandable member, wherein the retrieval bag is configured to receive a tissue specimen when the expandable member is in the expanded configuration.
